(19) United States
(12) Patent Application Publication
(10) Pub. No.: US 2014/0257465 A1
(43) Pub. Date: Sep. 11, 2014

(54) STENTS HAVING CONTROLLED ELUTION

(71) Applicant: MICELL TECHNOLOGIES, INC., Durham, NC (US)

(72) Inventors: James B. McClain, Raleigh, NC (US); Charles Douglas Taylor, Franklinton, NC (US); Wenda C. Carlyle, Prairie Farm, WI (US)

(21) Appl. No.: 14/238,677

(22) PCT Filed: Aug. 10, 2012
(86) PCT No.: PCT/US2012/050408

§ 371 Date: Jun. 2, 2014

Related U.S. Application Data
(60) Provisional application No. 61/523,210, filed on Aug. 12, 2011.
(60) Provisional application No. 61/556,742, filed on Nov. 7, 2011.
(60) Provisional application No. 61/581,057, filed on Dec. 28, 2011.
(60) Provisional application No. 61/623,469, filed on Apr. 12, 2012.

(51) Int. Cl. A61L 31/16; A61L 31/10; A61L 31/02; A61L 31/14

(52) U.S. Cl. CPC A61L 31/16; A61L 31/148; A61L 31/10; A61L 31/022; A61L 31/024; A61L 31/028
USPC 623/1.38; 623/1.42

(57) ABSTRACT

Provided herein is a device comprising a stent and a coating on the stent; wherein the coating comprises at least one polymer and at least one active agent; wherein at least part of the active agent is in crystalline form.

### CROSS REFERENCE

This application claims the benefit of U.S. Provisional Application No. 61/523,210, filed Aug. 12, 2011, U.S. Provisional Application No. 61/556,742, filed Nov. 7, 2011, U.S. Provisional Application No. 61/581,057, filed Dec. 28, 2011, and U.S. Provisional Application No. 61/623,469, filed Apr. 12, 2012, each of which the entire contents are incorporated herein by reference.

### BACKGROUND OF THE INVENTION

Drug-eluting stents are used to address the drawbacks of bare stents, namely to treat restenosis and to promote healing of the vessel after opening the blockage by PCI/stenting. Some current drug eluting stents can have physical, chemical and therapeutic legacy in the vessel over time. Others may have less legacy, but are not optimized for thickness, deployment flexibility, access to difficult lesions, and minimization of vessel wall intrusion.

### SUMMARY OF THE INVENTION

The present invention relates to methods for forming stents comprising a bioabsorbable polymer and a crystalline active agent in powder form onto a substrate.

It is desirable to have a drug-eluting stent with minimal physical, chemical and therapeutic legacy in the vessel after a proscribed period of time. This period of time is based on the effective healing of the vessel after opening the blockage by PCI/stenting (currently believed by leading clinicians to be 6-18 months).

It is also desirable to have drug-eluting stents of minimal cross-sectional thickness for (a) flexibility of deployment (b) access to small vessels and/or tortuous lesions (c) minimized intrusion into the vessel wall and blood.

Provided herein is a device comprising

a. a stent comprising a cobalt-chromium alloy; and

b. a coating on the stent; wherein the coating comprises at least one polymer and at least one crystalline active agent;
wherein the level of active agent degradation after two weeks incubation in a serum-supplemented cell culture medium at 37° C. is significantly reduced for the device as compared to a device comprising a metal cobalt-chromium stent and a coating comprising at least one polymer and at least one amorphous active agent.

Also provided herein is a device comprising

a. a stent comprising a cobalt-chromium alloy; and

b. a coating on the stent; wherein the coating comprises at least one polymer and at least one crystalline active agent;
wherein the coating disassociates from the stent following implantation of the device in a first artery of an animal and spreads within the vessel wall creating coating deposits in the neointima.

Also provided herein is a device comprising

a. a stent comprising a cobalt-chromium alloy; and

b. a coating on the stent; wherein the coating comprises at least one polymer and at least one crystalline active agent;
wherein there are, on average, fewer than twenty inflammatory cells associated with stent struts of the stent at 3 days following implantation of a single stent in a first artery of an animal.

Also provided herein is a device comprising

a. a first stent comprising a cobalt-chromium alloy; and

b. a coating on the first stent; wherein the coating comprises at least one polymer and at least one crystalline active agent;
wherein when said device is implanted in an overlapping manner with a second device in a first artery of an animal wherein the second device comprises

a. a second stent comprising a cobalt-chromium alloy; and

b. a coating on the second stent; wherein the coating comprises at least one polymer and at least one crystalline active agent;
there are, on average, fewer than twenty inflammatory cells associated with stent struts of the first stent at 3 days following implantation in the overlapping region of the overlapping devices.

In some embodiments, the crystalline active agent is at least one of: 50% crystalline, at least 75% crystalline, at least 90% crystalline. In certain embodiments, the crystalline active agent comprises pharmaceutical agent comprising at least one polymorph of the possible polymorphs of the crystalline structures of the pharmaceutical agent.

In certain embodiments, the polymer comprises a bioabsorbable polymer. In certain embodiments, the polymer comprises PLGA. In certain embodiments, the polymer comprises PLGA with a ratio of about 40:60 to about 60:40. In certain embodiments, the polymer comprises PLGA with a ratio of about 40:60 to about 60:40 and further comprises PLGA with a ratio of about 60:40 to about 90:10. In certain embodiments, the polymer comprises PLGA having a weight average molecular weight of about 25 kD. In certain embodiments, the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a weight average molecular weight of about 25 kD, a PLGA copolymer having a weight average molecular weight of about 31 kD, PGA poly(glycolide), LPLA poly(1-lactide), DLPLA poly(d1-lactide), PCL poly(ε-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof.

In certain embodiments, the stent comprises a cobalt-chromium alloy. In certain embodiments, the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co. In certain embodiments, the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co. In certain embodiments, the stent is formed from a material comprising a platinum chromium alloy or magnesium alloy. In certain embodiments, the stent is formed from a material comprising a platinum chromium alloy. In certain embodiments, the stent is formed from a material comprising a magnesium alloy. In some embodiments, the stent is fully absorbable or resorbable.

In some embodiments, the stent has a thickness of from about 50% to about 90% of a total thickness of the device. In certain embodiments, the coating has a total thickness of from about 5 μm to about 50 μm.

In some embodiments, the device has an active agent content of from about 5 μg to about 500 μg. In certain embodiments, the device has an active agent content of from about 100 μg to about 160 μg.

In some embodiments, the active agent comprises a macrolide immunosuppressive (limus) drug. In some embodiments, the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, myolimus, and salts, derivatives, isomers, racemates, diastereoisomers, prodrugs, hydrate, ester, or analogs thereof. As used herein, the terms “sirolimus” and “rapamycin” are interchangeable.

Provided herein is a method comprising
providing a coated stent comprising
   a stent comprising a cobalt-chromium alloy; and
   a coating on the stent; wherein the coating comprises at least one polymer and at least crystalline one active agent; and

wherein the level of active agent degradation after two weeks incubation in a serum-supplemented cell culture medium at 37° C. is significantly reduced for the device as compared to a device comprising a metal cobalt-chromium stent and a coating comprising at least one polymer and at least one amorphous active agent.

Provided herein is a method comprising
providing a coated stent comprising
   a stent comprising a cobalt-chromium alloy; and
   a coating on the stent; wherein the coating comprises at least one polymer and at least one crystalline active agent; and
implanting the coated stent in an animal,

wherein the coating disassociates from the stent following implantation of the device in a first artery of the animal and spreads within the vessel wall creating coating deposits in the neointima.

Provided herein is a method comprising
providing a coated stent comprising
   a stent comprising a cobalt-chromium alloy; and
   a coating on the stent; wherein the coating comprises at least one polymer and at least one crystalline active agent; and
implanting the coated stent in an animal,

wherein there are, on average, fewer than twenty inflammatory cells associated with stent struts of the stent at 3 days following implantation of a single stent in a first artery of the animal

Provided herein is a method comprising
providing a coated stent comprising
   a stent comprising a cobalt-chromium alloy; and
   a coating on the stent; wherein the coating comprises at least one polymer and at least one crystalline active agent; and
implanting the coated stent in an animal,

wherein when said device is implanted in an overlapping manner with a second device in a first artery of an animal wherein the second device comprises
a. a second stent comprising a cobalt-chromium alloy; and
b. a coating on the second stent; wherein the coating comprises at least one polymer and at least one crystalline active agent;
   there are, on average, fewer than twenty inflammatory cells associated with stent struts of the first stent at 3 days following implantation in the overlapping region of the overlapping devices.

In some embodiments of the method, the crystalline active agent is at least one of: 50% crystalline, at least 75% crystalline, at least 90% crystalline. In certain embodiments, the crystalline active agent comprises pharmaceutical agent comprising at least one polymorph of the possible polymorphs of the crystalline structures of the pharmaceutical agent.

In certain embodiments of the method, the polymer comprises a bioabsorbable polymer. In certain embodiments, the polymer comprises PLGA. In certain embodiments, the polymer comprises PLGA with a ratio of about 40:60 to about 60:40. In certain embodiments, the polymer comprises PLGA with a ratio of about 40:60 to about 60:40 and further comprises PLGA with a ratio of about 60:40 to about 90:10. In certain embodiments, the polymer comprises PLGA having a weight average molecular weight of about 25 kD. In certain embodiments, the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a weight average molecular weight of about 25 kD, a PLGA copolymer having a weight average molecular weight of about 31 kD, PGA poly(glycolide), LPLA poly(1-lactide), DLPLA poly(d1-lactide), PCL poly(ε-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof.

In certain embodiments of the method, the stent comprises a cobalt-chromium alloy. In certain embodiments, the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co. In certain embodiments, the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co. In certain embodiments, the stent is formed from a material comprising a platinum chromium alloy or magnesium alloy. In certain embodiments, the stent is formed from a material comprising a platinum chromium alloy. In certain embodiments, the stent is formed from a material comprising a magnesium alloy. In some embodiments, the stent is fully absorbable or resorbable.

In some embodiments of the method, the stent has a thickness of from about 50% to about 90% of a total thickness of the device. In certain embodiments, the coating has a total thickness of from about 5 μm to about 50 μm.

In some embodiments of the method, the device has an active agent content of from about 5 μg to about 500 μg. In certain embodiments, the device has an active agent content of from about 100 μg to about 160 μg.

In some embodiments of the method, the active agent comprises a macrolide immunosuppressive (limus) drug. In some embodiments, the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O—[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O—[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, myolimus, and salts, derivatives, isomers, racemates, diastereoisomers, prodrugs, hydrate, ester, or analogs thereof.

In some embodiments of the method, the active agent is a pharmaceutical agent. In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular phamaceutical agent herein which is at least in part crystalline, it is understood that any of the possible polymorphs of the pharmaceutical agent are contemplated.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts tissue from 30-day implantation of a Sirolimus DES of Example 2, at least, where the stent strut is visible marked as “S” and the clear spaces surrounding the strut (black arrows) represent areas previously occupied by coating that has been lost during processing of the histology slides; coating can be seen to have disassociated from the strut and spread into the surrounding tissue.

FIG. 1B depicts tissue from 30-day implantation of a Sirolimus DES of Example 2, at least, where the stent struts were lost during processing but their previous location is marked “S” and clear areas near strut location (arrows) represent coating that has disassociated from the strut and to become embedded in the neointima.

FIG. 2 depicts tissue from 90-day implantation of a Sirolimus DES of Example 2, at least, showing that by 90 days after implantation, there is no further evidence of coating deposits suggesting near complete disassociation of the coating from the stent strut by that point.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments contemplated herein will be apparent to those skilled in the art in light of the instant disclosure, which do not depart from the instant invention. Hence, the following specification is intended to illustrate selected embodiments of the invention, and not to exhaustively specify all permutations, combinations and variations thereof.

### DEFINITIONS

As used in the present specification, the following words and phrases are generally intended to have the meanings as set forth below, except to the extent that the context in which they are used indicates otherwise.

“Substrate” as used herein, refers to any surface upon which it is desirable to deposit a coating comprising a polymer and a pharmaceutical or biological agent, wherein the coating process does not substantially modify the morphology of the pharmaceutical agent or the activity of the biological agent. Biomedical implants are of particular interest for the present invention; however the present invention is not intended to be restricted to this class of substrates. Those of skill in the art will appreciate alternate substrates that could benefit from the coating process described herein, such as pharmaceutical tablet cores, as part of an assay apparatus or as components in a diagnostic kit (e.g. a test strip).

“Biomedical implant” as used herein refers to any implant for insertion into the body of a human or animal subject, including but not limited to stents (e.g., coronary stents, vascular stents including peripheral stents and graft stents, urinary tract stents, urethral/prostatic stents, rectal stent, oesophageal stent, biliary stent, pancreatic stent), electrodes, catheters, leads, implantable pacemaker, cardioverter or defibrillator housings, joints, screws, rods, ophthalmic implants, femoral pins, bone plates, grafts, anastomotic devices, perivascular wraps, sutures, staples, shunts for hydrocephalus, dialysis grafts, colostomy bag attachment devices, ear drainage tubes, leads for pace makers and implantable cardioverters and defibrillators, vertebral disks, bone pins, suture anchors, hemostatic barriers, clamps, screws, plates, clips, vascular implants, tissue adhesives and sealants, tissue scaffolds, various types of dressings (e.g., wound dressings), bone substitutes, intraluminal devices, vascular supports, etc.

The implants may be formed from any suitable material, including but not limited to polymers (including stable or inert polymers, organic polymers, organic-inorganic copolymers, inorganic polymers, and biodegradable polymers), metals, metal alloys, inorganic materials such as silicon, and composites thereof, including layered structures with a core of one material and one or more coatings of a different material. Substrates made of a conducting material facilitate electrostatic capture. However, the invention contemplates the use of electrostatic capture, as described below, in conjunction with substrate having low conductivity or which are non-conductive. To enhance electrostatic capture when a non-conductive substrate is employed, the substrate is processed for example while maintaining a strong electrical field in the vicinity of the substrate.

Subjects into which biomedical implants of the invention may be applied or inserted include both human subjects (including male and female subjects and infant, juvenile, adolescent, adult and geriatric subjects) as well as animal subjects (including but not limited to pig, rabbit, mouse, dog, cat, horse, monkey, etc.) for veterinary purposes and/or medical research.

In a preferred embodiment the biomedical implant is an expandable intraluminal vascular graft or stent that can be expanded within a blood vessel by an angioplasty balloon associated with a catheter to dilate and expand the lumen of a blood vessel, such as described in U.S. Pat. No. 4,733,665 to Palmaz.

“Pharmaceutical agent” as used herein refers to any of a variety of drugs or pharmaceutical compounds that can be used as active agents to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the pharmaceutical agents of the invention may also comprise two or more drugs or pharmaceutical compounds. Pharmaceutical agents, include but are not limited to antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals, chemotherapeutic agents and amino acids. Examples of suitable active ingredients are acarbose, antigens, beta-receptor blockers, non-steroidal antiinflammatory drugs [NSAIDs], cardiac glycosides, acetylsalicylic acid, virustatics, aclarubicin, acyclovir, cisplatin, actinomycin, alpha- and beta-sympatomimetics, (dimeprazole, allopurinol, alprostadil, prostaglandins, amantadine, ambroxol, amlodipine, methotrexate, S-aminosalicylic acid, amitriptyline, amoxicillin, anastrozole, atenolol, azathioprine, balsalazide, beclomethasone, betahistine, bezafibrate, bicalutamide, diazepam and diazepam derivatives, budesonide, bufexamac, buprenorphine, methadone, calcium salts, potassium salts, magnesium salts, candesartan, carbamazepine, captopril, cefalosporins, cetirizine, chenodeoxycholic acid, ursodeoxycholic acid, theophylline and theophylline derivatives, trypsins, cimetidine, clarithromycin, clavulanic acid, clindamycin, clobutinol, clonidine, cotrimoxazole, codeine, caffeine, vitamin D and derivatives of vitamin D, colestyramine, cromoglicic acid, coumarin and coumarin derivatives, cysteine, cytarabine, cyclophosphamide, ciclosporin, cyproterone, cytabarine, dapiprazole, desogestrel, desonide, dihydralazine, diltiazem, ergot alkaloids, dimenhydrinate, dimethyl sulphoxide, dimethicone, domperidone and domperidan derivatives, dopamine, doxazosin, doxorubicin, doxylamine, dapiprazole, benzodiazepines, diclofenac, glycoside antibiotics, desipramine, econazole, ACE inhibitors, enalapril, ephedrine, epinephrine, epoetin and epoetin derivatives, morphinans, calcium antagonists, irinotecan, modafinil, orlistat, peptide antibiotics, phenyloin, riluzoles, risedronate, sildenafil, topiramate, macrolide antibiotics, oestrogen and oestrogen derivatives, progestogen and progestogen derivatives, testosterone and testosterone derivatives, androgen and androgen derivatives, ethenzamide, etofenamate, etofibrate, fenofibrate, etofylline, etoposide, famciclovir, famotidine, felodipine, fenofibrate, fentanyl, fenticonazole, gyrase inhibitors, fluconazole, fludarabine, fluarizine, fluorouracil, fluoxetine, flurbiprofen, ibuprofen, flutamide, fluvastatin, follitropin, formoterol, fosfomicin, furosemide, fusidic acid, gallopamil, ganciclovir, gemfibrozil, gentamicin, ginkgo, Saint John's wort, glibenclamide, urea derivatives as oral antidiabetics, glucagon, glucosamine and glucosamine derivatives, glutathione, glycerol and glycerol derivatives, hypothalamus hormones, goserelin, gyrase inhibitors, guanethidine, halofantrine, haloperidol, heparin and heparin derivatives, hyaluronic acid, hydralazine, hydrochlorothiazide and hydrochlorothiazide derivatives, salicylates, hydroxyzine, idarubicin, ifosfamide, imipramine, indometacin, indoramine, insulin, interferons, iodine and iodine derivatives, isoconazole, isoprenaline, glucitol and glucitol derivatives, itraconazole, ketoconazole, ketoprofen, ketotifen, lacidipine, lansoprazole, levodopa, levomethadone, thyroid hormones, lipoic acid and lipoic acid derivatives, lisinopril, lisuride, lofepramine, lomustine, loperamide, loratadine, maprotiline, mebendazole, mebeverine, meclozine, mefenamic acid, mefloquine, meloxicam, mepindolol, meprobamate, meropenem, mesalazine, mesuximide, metamizole, metformin, methotrexate, methylphenidate, methylprednisolone, metixene, metoclopramide, metoprolol, metronidazole, mianserin, miconazole, minocycline, minoxidil, misoprostol, mitomycin, mizolastine, moexipril, morphine and morphine derivatives, evening primrose, nalbuphine, naloxone, tilidine, naproxen, narcotine, natamycin, neostigmine, nicergoline, nicethamide, nifedipine, niflumic acid, nimodipine, nimorazole, nimustine, nisoldipine, adrenaline and adrenaline derivatives, norfloxacin, novamine sulfone, noscapine, nystatin, ofloxacin, olanzapine, olsalazine, omeprazole, omoconazole, ondansetron, oxaceprol, oxacillin, oxiconazole, oxymetazoline, pantoprazole, paracetamol, paroxetine, penciclovir, oral penicillins, pentazocine, pentifylline, pentoxifylline, perphenazine, pethidine, plant extracts, phenazone, pheniramine, barbituric acid derivatives, phenylbutazone, phenyloin, pimozide, pindolol, piperazine, piracetam, pirenzepine, piribedil, piroxicam, pramipexole, pravastatin, prazosin, procaine, promazine, propiverine, propranolol, propyphenazone, prostaglandins, protionamide, proxyphylline, quetiapine, quinapril, quinaprilat, ramipril, ranitidine, reproterol, reserpine, ribavirin, rifampicin, risperidone, ritonavir, ropinirole, roxatidine, roxithromycin, ruscogenin, rutoside and rutoside derivatives, sabadilla, salbutamol, salmeterol, scopolamine, selegiline, sertaconazole, sertindole, sertralion, silicates, sildenafil, simvastatin, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulfasalazine, sulpiride, sultamicillin, sultiam, sumatriptan, suxamethonium chloride, tacrine, tacrolimus, taliolol, tamoxifen, taurolidine, tazarotene, temazepam, teniposide, tenoxicam, terazosin, terbinafine, terbutaline, terfenadine, terlipressin, tertatolol, tetracyclins, teryzoline, theobromine, theophylline, butizine, thiamazole, phenothiazines, thiotepa, tiagabine, tiapride, propionic acid derivatives, ticlopidine, timolol, timidazole, tioconazole, tioguanine, tioxolone, tiropramide, tizanidine, tolazoline, tolbutamide, tolcapone, tolnaftate, tolperisone, topotecan, torasemide, antioestrogens, tramadol, tramazoline, trandolapril, tranylcypromine, trapidil, trazodone, triamcinolone and triamcinolone derivatives, triamterene, trifluperidol, trifluridine, trimethoprim, trimipramine, tripelennamine, triprolidine, trifosfamide, tromantadine, trometamol, tropalpin, troxerutine, tulobuterol, tyramine, tyrothricin, urapidil, ursodeoxycholic acid, chenodeoxycholic acid, valaciclovir, valproic acid, vancomycin, vecuronium chloride, Viagra, venlafaxine, verapamil, vidarabine, vigabatrin, viloazine, vinblastine, vincamine, vincristine, vindesine, vinorelbine, vinpocetine, viquidil, warfarin, xantinol nicotinate, xipamide, zafirlukast, zalcitabine, zidovudine, zolmitriptan, zolpidem, zoplicone, zotipine and the like. See, e.g., U.S. Pat. No. 6,897,205; see also U.S. Pat. No. 6,838,528; U.S. Pat. No. 6,497,729, incorporated herein by reference in their entirety.

Examples of therapeutic agents employed in conjunction with the invention include, rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(25)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

As used herein, the pharmaceutical agent sirolimus may also and/or alternatively be called rapamycin, or vice versa, unless otherwise noted with regard to a particular term—for nonlimiting example, 42-Epi-(tetrazolyl)rapamycin is tacrolimus as noted herein.

The pharmaceutical agents may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers. As well, the pharmaceutical agent may include a prodrug, a hydrate, an ester, a derivative or analogs of a compound or molecule.

In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular pharmaceutical agent herein which is at least in part crystalline, it is understood that any of the possible polymorphs of the pharmaceutical agent are contemplated.

A “pharmaceutically acceptable salt” may be prepared for any pharmaceutical agent having a functionality capable of forming a salt, for example an acid or base functionality. Pharmaceutically acceptable salts may be derived from organic or inorganic acids and bases. The term “pharmaceutically-acceptable salts” in these instances refers to the relatively non-toxic, inorganic and organic base addition salts of the pharmaceutical agents.

“Prodrugs” are derivative compounds derivatized by the addition of a group that endows greater solubility to the compound desired to be delivered. Once in the body, the prodrug is typically acted upon by an enzyme, e.g., an esterase, amidase, or phosphatase, to generate the active compound.

“Stability” as used herein in refers to the stability of the drug in a polymer coating deposited on a substrate in its final product form (e.g., stability of the drug in a coated stent). The term stability will define 5% or less degradation of the drug in the final product form.

“Active biological agent” as used herein refers to a substance, originally produced by living organisms, that can be used to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the active biological agents of the invention may also comprise two or more active biological agents or an active biological agent combined with a pharmaceutical agent, a stabilizing agent or chemical or biological entity. Although the active biological agent may have been originally produced by living organisms, those of the present invention may also have been synthetically prepared, or by methods combining biological isolation and synthetic modification. By way of a non-limiting example, a nucleic acid could be isolated form from a biological source, or prepared by traditional techniques, known to those skilled in the art of nucleic acid synthesis. Furthermore, the nucleic acid may be further modified to contain non-naturally occurring moieties. Non-limiting examples of active biological agents include peptides, proteins, enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates and the like. They further include, but are not limited to, antirestenotic agents, antidiabetics, analgesics, antiinflammatory agents, antirheumatics, antihypotensive agents, antihypertensive agents, psychoactive drugs, tranquillizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals and chemotherapeutic agents. Preferably, the active biological agent is a peptide, protein or enzyme, including derivatives and analogs of natural peptides, proteins and enzymes. The active biological agent may also be a hormone, gene therapies, RNA, siRNA, and/or cellular therapies (for non-limiting example, stem cells or T-cells).

“Active agent” as used herein refers to any pharmaceutical agent or active biological agent as described herein.

“Activity” as used herein refers to the ability of a pharmaceutical or active biological agent to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). Thus the activity of a pharmaceutical or active biological agent should be of therapeutic or prophylactic value.

“Secondary, tertiary and quaternary structure” as used herein are defined as follows. The active biological agents of the present invention will typically possess some degree of secondary, tertiary and/or quaternary structure, upon which the activity of the agent depends. As an illustrative, non-limiting example, proteins possess secondary, tertiary and quaternary structure. Secondary structure refers to the spatial arrangement of amino acid residues that are near one another in the linear sequence. The α-helix and the β-strand are elements of secondary structure. Tertiary structure refers to the spatial arrangement of amino acid residues that are far apart in the linear sequence and to the pattern of disulfide bonds. Proteins containing more than one polypeptide chain exhibit an additional level of structural organization. Each polypeptide chain in such a protein is called a subunit. Quaternary structure refers to the spatial arrangement of subunits and the nature of their contacts. For example hemoglobin consists of two α and two β chains. It is well known that protein function arises from its conformation or three dimensional arrangement of atoms (a stretched out polypeptide chain is devoid of activity). Thus one aspect of the present invention is to manipulate active biological agents, while being careful to maintain their conformation, so as not to lose their therapeutic activity.

“Polymer” as used herein, refers to a series of repeating monomeric units that have been cross-linked or polymerized. Any suitable polymer can be used to carry out the present invention. It is possible that the polymers of the invention may also comprise two, three, four or more different polymers. In some embodiments, of the invention only one polymer is used. In some preferred embodiments a combination of two polymers are used. Combinations of polymers can be in varying ratios, to provide coatings with differing properties. Those of skill in the art of polymer chemistry will be familiar with the different properties of polymeric compounds.

Polymers useful in the devices and methods of the present invention include, for example, stable polymers, biostable polymers, durable polymers, inert polymers, organic polymers, organic-inorganic copolymers, inorganic polymers, bioabsorbable, bioresorbable, resorbable, degradable, and biodegradable polymers. These categories of polymers may, in some cases, be synonymous, and is some cases may also and/or alternatively overlap. Those of skill in the art of polymer chemistry will be familiar with the different properties of polymeric compounds.

In some embodiments, the coating comprises a polymer. In some embodiments, the active agent comprises a polymer. In some embodiments, the polymer comprises at least one of polyalkyl methacrylates, polyalkylene-co-vinyl acetates, polyalkylenes, polyurethanes, polyanhydrides, aliphatic polycarbonates, polyhydroxyalkanoates, silicone containing polymers, polyalkyl siloxanes, aliphatic polyesters, polyglycolides, polylactides, polylactide-co-glycolides, poly(e-caprolactone)s, polytetrahalooalkylenes, polystyrenes, poly(phosphasones), copolymers thereof, and combinations thereof.

Examples of polymers that may be used in the present invention include, but are not limited to polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropytenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof.

The polymers of the present invention may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene)—and derivatives and copolymers such as those commonly sold as Teflon(R) products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Examples of polymers that may be used in the present invention include, but are not limited to polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropytenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof.

The polymers of the present invention may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene)—and derivatives and copolymers such as those commonly sold as Teflon(R) products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Suitable polymers also include absorbable and/or resorbable polymers including the following, combinations, copolymers and derivatives of the following: Polylactides (PLA), Polyglycolides (PGA), PolyLactide-co-glycolides (PLGA), Polyanhydrides, Polyorthoesters, Poly(N-(2-hydroxypropyl) methacrylamide), Poly(1-aspartamide), including the derivatives DLPLA—poly(d1-lactide); LPLA—poly(1-lactide); PDO—poly(dioxanone); PGA-TMC—poly(glycolide-co-trimethylene carbonate); PGA-LPLA—poly(1-lactide-co-glycolide); PGA-DLPLA—poly(d1-lactide-co-glycolide); LPLA-DLPLA—poly(1-lactide-co-d1-lactide); and PDO-PGA-TMC—poly(glycolide-co-trimethylene carbonate-co-dioxanone), and combinations thereof.

“Copolymer” as used herein refers to a polymer being composed of two or more different monomers. A copolymer may also and/or alternatively refer to random, block, graft, copolymers known to those of skill in the art.

“Biocompatible” as used herein, refers to any material that does not cause injury or death to the animal or induce an adverse reaction in an animal when placed in intimate contact with the animal's tissues. Adverse reactions include for example inflammation, infection, fibrotic tissue formation, cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, causes inflammation or irritation, or induces an immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner the sample is degraded in 1 M NaOH at 37 degrees C. until complete degradation is observed. The solution is then neutralized with 1 M HCl. About 200 microliters of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The terms “bioabsorbable,” “biodegradable,” “bioerodible,” and “bioresorbable,” are art-recognized synonyms. These terms are used herein interchangeably. Bioabsorbable polymers typically differ from non-bioabsorbable polymers (i.e. durable polymers) in that the former may be absorbed (e.g.; degraded) during use. In certain embodiments, such use involves in vivo use, such as in vivo therapy, and in other certain embodiments, such use involves in vitro use. In general, degradation attributable to biodegradability involves the degradation of a bioabsorbable polymer into its component subunits, or digestion, e.g., by a biochemical process, of the polymer into smaller, non-polymeric subunits. In certain embodiments, biodegradation may occur by enzymatic mediation, degradation in the presence of water (hydrolysis) and/or other chemical species in the body, or both. The bioabsorbabilty of a polymer may be shown in-vitro as described herein or by methods known to one of skill in the art. An in-vitro test for bioabsorbability of a polymer does not require living cells or other biologic materials to show bioabsorption properties (e.g. degradation, digestion). Thus, resorbtion, resorption, absorption, absorbtion, erosion may also be used synonymously with the terms “bioabsorbable,” “biodegradable,” “bioerodible,” and “bioresorbable.” Mechanisms of degradation of a bioabsorbable polymer may include, but are not limited to, bulk degradation, surface erosion, and combinations thereof.

As used herein, the term “biodegradation” encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the biostability, the biodegradation of any bioabsorbable polymer is usually slower.

As used herein, the term “durable polymer” refers to a polymer that is not bioabsorbable (and/or is not bioerodable, and/or is not biodegradable, and/or is not bioresorbable) and is, thus biostable. In some embodiments, the device comprises a durable polymer. The polymer may include a cross-linked durable polymer. Example biocompatible durable polymers include, but are not limited to: polyester, aliphatic polyester, polyanhydride, polyethylene, polyorthoester, polyphosphazene, polyurethane, polycarbonate urethane, aliphatic polycarbonate, silicone, a silicone containing polymer, polyolefin, polyamide, polycaprolactam, polyamide, polyvinyl alcohol, acrylic polymer, acrylate, polystyrene, epoxy, polyethers, cellulosics, expanded polytetrafluoroethylene, phosphorylcholine, polyethyleneyerphthalate, polymethylmethavrylate, poly(ethylmethacrylate/n-butylmethacrylate), parylene C, polyethylene-co-vinyl acetate, polyalkyl methacrylates, polyalkylene-co-vinyl acetate, polyalkylene, polyalkyl siloxanes, polyhydroxyalkanoate, polyfluoroalkoxyphasphazine, poly(styrene-b-isobutylene-b-styrene), poly-butyl methacrylate, poly-byta-diene, and blends, combinations, homopolymers, condensation polymers, alternating, block, dendritic, crosslinked, and copolymers thereof. The polymer may include a thermoset material. The polymer may provide strength for the coated implantable medical device. The polymer may provide durability for the coated implantable medical device. The coatings and coating methods provided herein provide substantial protection from these by establishing a multi-layer coating which can be bioabsorbable or durable or a combination thereof, and which can both deliver active agents and provide elasticity and radial strength for the vessel in which it is delivered.

“Therapeutically desirable morphology” as used herein refers to the gross form and structure of the pharmaceutical agent, once deposited on the substrate, so as to provide for optimal conditions of ex vivo storage, in vivo preservation and/or in vivo release. Such optimal conditions may include, but are not limited to increased shelf life, increased in vivo stability, good biocompatibility, good bioavailability or modified release rates. Typically, for the present invention, the desired morphology of a pharmaceutical agent would be crystalline or semi-crystalline or amorphous, although this may vary widely depending on many factors including, but not limited to, the nature of the pharmaceutical agent, the disease to be treated/prevented, the intended storage conditions for the substrate prior to use or the location within the body of any biomedical implant. Preferably at least 10%, 20%, 30%, 40%, 50%, 60%, 70%, 80%, 90% or 100% of the pharmaceutical agent is in crystalline or semi-crystalline form.

“Stabilizing agent” as used herein refers to any substance that maintains or enhances the stability of the biological agent. Ideally these stabilizing agents are classified as Generally Regarded As Safe (GRAS) materials by the US Food and Drug Administration (FDA). Examples of stabilizing agents include, but are not limited to carrier proteins, such as albumin, gelatin, metals or inorganic salts. Pharmaceutically acceptable excipient that may be present can further be found in the relevant literature, for example in the Handbook of Pharmaceutical Additives: An International Guide to More Than 6000 Products by Trade Name, Chemical, Function, and Manufacturer; Michael and Irene Ash (Eds.); Gower Publishing Ltd.; Aldershot, Hampshire, England, 1995.

“Compressed fluid” as used herein refers to a fluid of appreciable density (e.g., >0.2 g/cc) that is a gas at standard temperature and pressure. “Supercritical fluid”, “near-critical fluid”, “near-supercritical fluid”, “critical fluid”, “densified fluid” or “densified gas” as used herein refers to a compressed fluid under conditions wherein the temperature is at least 80% of the critical temperature of the fluid and the pressure is at least 50% of the critical pressure of the fluid, and/or a density of +50% of the critical density of the fluid.

Examples of substances that demonstrate supercritical or near critical behavior suitable for the present invention include, but are not limited to carbon dioxide, isobutylene, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydrofluorocarbons, perfluorocarbons (such as perfluoromethane and perfluoropropane, chloroform, trichloro-fluoromethane, dichloro-difluoromethane, dichloro-tetrafluoroethane) and mixtures thereof. Preferably, the supercritical fluid is hexafluoropropane (FC-236EA), or 1,1,1,2,3,3-hexafluoropropane. Preferably, the supercritical fluid is hexafluoropropane (FC-236EA), or 1,1,1,2,3,3-hexafluoropropane for use in PLGA polymer coatings.

“Sintering” as used herein refers to the process by which parts of the polymer or the entire polymer becomes continuous (e.g., formation of a continuous polymer film). As discussed below, the sintering process is controlled to produce a fully conformal continuous polymer (complete sintering) or to produce regions or domains of continuous coating while producing voids (discontinuities) in the polymer. As well, the sintering process is controlled such that some phase separation is obtained or maintained between polymer different polymers (e.g., polymers A and B) and/or to produce phase separation between discrete polymer particles. Through the sintering process, the adhesions properties of the coating are improved to reduce flaking of detachment of the coating from the substrate during manipulation in use. As described below, in some embodiments, the sintering process is controlled to provide incomplete sintering of the polymer. In embodiments involving incomplete sintering, a polymer is formed with continuous domains, and voids, gaps, cavities, pores, channels or, interstices that provide space for sequestering a therapeutic agent which is released under controlled conditions. Depending on the nature of the polymer, the size of polymer particles and/or other polymer properties, a compressed gas, a densified gas, a near critical fluid or a super-critical fluid may be employed. In one example, carbon dioxide is used to treat a substrate that has been coated with a polymer and a drug, using dry powder and RESS electrostatic coating processes. In another example, isobutylene is employed in the sintering process. In other examples a mixture of carbon dioxide and isobutylene is employed. In another example, 1,1,2,3,3-hexafluoropropane is employed in the sintering process.

When an amorphous material is heated to a temperature above its glass transition temperature, or when a crystalline material is heated to a temperature above a phase transition temperature, the molecules comprising the material are more mobile, which in turn means that they are more active and thus more prone to reactions such as oxidation. However, when an amorphous material is maintained at a temperature below its glass transition temperature, its molecules are substantially immobilized and thus less prone to reactions. Likewise, when a crystalline material is maintained at a temperature below its phase transition temperature, its molecules are substantially immobilized and thus less prone to reactions. Accordingly, processing drug components at mild conditions, such as the deposition and sintering conditions described herein, minimizes cross-reactions and degradation of the drug component. One type of reaction that is minimized by the processes of the invention relates to the ability to avoid conventional solvents which in turn minimizes—oxidation of drug, whether in amorphous, semi-crystalline, or crystalline form, by reducing exposure thereof to free radicals, residual solvents, protic materials, polar-protic materials, oxidation initiators, and autoxidation initiators.

“Rapid Expansion of Supercritical Solutions” or “RESS” as used herein involves the dissolution of a polymer into a compressed fluid, typically a supercritical fluid, followed by rapid expansion into a chamber at lower pressure, typically near atmospheric conditions. The rapid expansion of the supercritical fluid solution through a small opening, with its accompanying decrease in density, reduces the dissolution capacity of the fluid and results in the nucleation and growth of polymer particles. The atmosphere of the chamber is maintained in an electrically neutral state by maintaining an isolating “cloud” of gas in the chamber. Carbon dioxide, nitrogen, argon, helium, or other appropriate gas is employed to prevent electrical charge is transferred from the substrate to the surrounding environment.

“Bulk properties” properties of a coating including a pharmaceutical or a biological agent that can be enhanced through the methods of the invention include for example: adhesion, smoothness, conformality, thickness, and compositional mixing.

“Electrostatically charged” or “electrical potential” or “electrostatic capture” or “e-” as used herein refers to the collection of the spray-produced particles upon a substrate that has a different electrostatic potential than the sprayed particles. Thus, the substrate is at an attractive electronic potential with respect to the particles exiting, which results in the capture of the particles upon the substrate. i.e. the substrate and particles are oppositely charged, and the particles transport through the gaseous medium of the capture vessel onto the surface of the substrate is enhanced via electrostatic attraction. This may be achieved by charging the particles and grounding the substrate or conversely charging the substrate and grounding the particles, by charging the particles at one potential (e.g. negative charge) and charging the substrate at an opposite potential (e.g. positive charge), or by some other process, which would be easily envisaged by one of skill in the art of electrostatic capture.

“Intimate mixture” as used herein, refers to two or more materials, compounds, or substances that are uniformly distributed or dispersed together.

“Layer” as used herein refers to a material covering a surface or forming an overlying part or segment. Two different layers may have overlapping portions whereby material from one layer may be in contact with material from another layer. Contact between materials of different layers can be measured by determining a distance between the materials. For example, Raman spectroscopy may be employed in identifying materials from two layers present in close proximity to each other.

While layers defined by uniform thickness and/or regular shape are contemplated herein, several embodiments described below relate to layers having varying thickness and/or irregular shape. Material of one layer may extend into the space largely occupied by material of another layer. For example, in a coating having three layers formed in sequence as a first polymer layer, a pharmaceutical agent layer and a second polymer layer, material from the second polymer layer which is deposited last in this sequence may extend into the space largely occupied by material of the pharmaceutical agent layer whereby material from the second polymer layer may have contact with material from the pharmaceutical layer. It is also contemplated that material from the second polymer layer may extend through the entire layer largely occupied by pharmaceutical agent and contact material from the first polymer layer.

It should be noted however that contact between material from the second polymer layer (or the first polymer layer) and material from the pharmaceutical agent layer (e.g.; a pharmaceutical agent crystal particle or a portion thereof) does not necessarily imply formation of a mixture between the material from the first or second polymer layers and material from the pharmaceutical agent layer. In some embodiments, a layer may be defined by the physical three-dimensional space occupied by crystalline particles of a pharmaceutical agent (and/or biological agent). It is contemplated that such layer may or may not be continuous as physical space occupied by the crystal particles of pharmaceutical agents may be interrupted, for example, by polymer material from an adjacent polymer layer. An adjacent polymer layer may be a layer that is in physical proximity to be pharmaceutical agent particles in the pharmaceutical agent layer. Similarly, an adjacent layer may be the layer formed in a process step right before or right after the process step in which pharmaceutical agent particles are deposited to form the pharmaceutical agent layer.

As described below, material deposition and layer formation provided herein are advantageous in that the pharmaceutical agent remains largely in crystalline form during the entire process. While the polymer particles and the pharmaceutical agent particles may be in contact, the layer formation process is controlled to avoid formation of a mixture between the pharmaceutical agent particles the polymer particles during formation of a coated device.

“Laminate coating” as used herein refers to a coating made up of two or more layers of material. Means for creating a laminate coating as described herein (e.g.; a laminate coating comprising bioabsorbable polymer(s) and pharmaceutical agent) may include coating the stent with drug and polymer as described herein (e-RESS, e-DPC, compressed-gas sintering). The process comprises performing multiple and sequential coating steps (with sintering steps for polymer materials) wherein different materials may be deposited in each step, thus creating a laminated structure with a multitude of layers (at least 2 layers) including polymer layers and pharmaceutical agent layers to build the final device (e.g.; laminate coated stent).

The coating methods provided herein may be calibrated to provide a coating bias whereby the mount of polymer and pharmaceutical agent deposited in the abluminal surface of the stent (exterior surface of the stent) is greater than the amount of pharmaceutical agent and amount of polymer deposited on the luminal surface of the stent (interior surface of the stent). The resulting configuration may be desirable to provide preferential elution of the drug toward the vessel wall (luminal surface of the stent) where the therapeutic effect of anti-restenosis is desired, without providing the same antiproliferative drug(s) on the abluminal surface, where they may retard healing, which in turn is suspected to be a cause of late-stage safety problems with current DESs.

As well, the methods described herein provide a device wherein the coating on the stent is biased in favor of increased coating at the ends of the stent. For example, a stent having three portions along the length of the stent (e.g.; a central portion flanked by two end portions) may have end portions coated with increased amounts of pharmaceutical agent and/or polymer compared to the central portion.

The present invention provides numerous advantages. The invention is advantageous in that it allows for employing a platform combining layer formation methods based on compressed fluid technologies; electrostatic capture and sintering methods. The platform results in drug eluting stents having enhanced therapeutic and mechanical properties. The invention is particularly advantageous in that it employs optimized laminate polymer technology. In particular, the present invention allows the formation of discrete layers of specific drug platforms. As indicated above, the shape of a discrete layer of crystal particles may be irregular, including interruptions of said layer by material from another layer (polymer layer) positioned in space between crystalline particles of pharmaceutical agent.

Conventional processes for spray coating stents require that drug and polymer be dissolved in solvent or mutual solvent before spray coating can occur. The platform provided herein the drugs and polymers are coated on the stent framework in discrete steps, which can be carried out simultaneously or alternately. This allows discrete deposition of the active agent (e.g., a drug) within a polymer thereby allowing the placement of more than one drug on a single medical device with or without an intervening polymer layer. For example, the present platform provides a dual drug eluting stent.

Some of the advantages provided by the subject invention include employing compressed fluids (e.g., supercritical fluids, for example E-RESS based methods); solvent free deposition methodology; a platform that allows processing at lower temperatures thereby preserving the qualities of the active agent and the polymer; the ability to incorporate two, three or more drugs while minimizing deleterious effects from direct interactions between the various drugs and/or their excipients during the fabrication and/or storage of the drug eluting stents; a dry deposition; enhanced adhesion and mechanical properties of the layers on the stent framework; precision deposition and rapid batch processing; and ability to form intricate structures.

In one embodiment, the present invention provides a multi-drug delivery platform which produces strong, resilient and flexible drug eluting stents including an anti-restenosis drug (e.g., a limus or taxol) and anti-thrombosis drug (e.g., heparin or an analog thereof) and well characterized bioabsorbable polymers. The drug eluting stents provided herein minimize potential for thrombosis, in part, by reducing or totally eliminating thrombogenic polymers and reducing or totally eliminating residual drugs that could inhibit healing.

The platform provides optimized delivery of multiple drug therapies for example for early stage treatment (restenosis) and late-stage (thrombosis).

The platform also provides an adherent coating which enables access through tortuous lesions without the risk of the coating being compromised.

Another advantage of the present platform is the ability to provide highly desirable eluting profiles.

Advantages of the invention include the ability to reduce or completely eliminate potentially thrombogenic polymers as well as possibly residual drugs that may inhibit long term healing. As well, the invention provides advantageous stents having optimized strength and resilience if coatings which in turn allows access to complex lesions and reduces or completely eliminates delamination. Laminated layers of bioabsorbable polymers allow controlled elution of one or more drugs.

The platform provided herein reduces or completely eliminates shortcoming that have been associated with conventional drug eluting stents. For example, the platform provided herein allows for much better tuning of the period of time for the active agent to elute and the period of time necessary for the polymer to resorb thereby minimizing thrombosis and other deleterious effects associate with poorly controlled drug release.

The present invention provides several advantages which overcome or attenuate the limitations of current technology for bioabsorbable stents. For example, an inherent limitation of conventional bioabsorbable polymeric materials relates to the difficulty in forming to a strong, flexible, deformable (e.g. balloon deployable) stent with low profile. The polymers generally lack the strength of high-performance metals. The present invention overcomes these limitations by creating a laminate structure in the essentially polymeric stent. Without wishing to be bound by any specific theory or analogy, the increased strength provided by the stents of the invention can be understood by comparing the strength of plywood vs. the strength of a thin sheet of wood.

Embodiments of the invention involving a thin metallic stent-framework provide advantages including the ability to overcome the inherent elasticity of most polymers. It is generally difficult to obtain a high rate (e.g., 100%) of plastic deformation in polymers (compared to elastic deformation where the materials have some ‘spring back’ to the original shape). Again, without wishing to be bound by any theory, the central metal stent framework (that would be too small and weak to serve as a stent itself) would act like wires inside of a plastic, deformable stent, basically overcoming any ‘elastic memory’ of the polymer.

Another advantage of the present invention is the ability to create a stent with a controlled (dialed-in) drug-elution profile. Via the ability to have different materials in each layer of the laminate structure and the ability to control the location of drug(s) independently in these layers, the method enables a stent that could release drugs at very specific elution profiles, programmed sequential and/or parallel elution profiles. Also, the present invention allows controlled elution of one drug without affecting the elution of a second drug (or different doses of the same drug).

Provided herein is a device comprising a stent; and a coating on the stent; wherein the coating comprises at least one bioabsorbable polymer and at least one active agent; wherein the active agent is present in crystalline form on at least one region of an outer surface of the coating opposite the stent and wherein 50% or less of the total amount of active agent in the coating is released after 24 hours in vitro elution.

In some embodiments, in vitro elution is carried out in a 1:1 spectroscopic grade ethanol (95%)/phosphate buffer saline at pH 7.4 and 37° C.; wherein the amount of active agent released is determined by measuring UV absorption. In some embodiments, UV absorption is detected at 278 nm by a diode array spectrometer.

In some embodiments, in vitro elution testing, and/or any other test method described herein is performed following the final sintering step. In some embodiments, in vitro elution testing, and/or any other test method described herein is performed prior to crimping the stent to a balloon catheter. In some embodiments, in vitro elution testing, and/or any other test method described herein is performed following sterilization. In some embodiments in vitro elution testing, and/or any other test method described herein is performed following crimping the stent to a balloon catheter. In some embodiments, in vitro elution testing, and/or any other test method described herein is performed following expansion of the stent to nominal pressure of the balloon onto which the stent has been crimped. In some embodiments, in vitro elution testing, and/or any other test method described herein is performed following expansion of the stent to the rated burst pressure of the balloon to which the stent has been crimped.

In some embodiments, presence of active agent on at least a region of the surface of the coating is determined by cluster secondary ion mass spectrometry (cluster SIMS). In some embodiments, presence of active agent on at least a region of the surface of the coating is determined by generating cluster secondary ion mass spectrometry (cluster SIMS) depth profiles. In some embodiments, presence of active agent on at least a region of the surface of the coating is determined by time of flight secondary ion mass spectrometry (TOF-SIMS). In some embodiments, presence of active agent on at least a region of the surface of the coating is determined by atomic force microscopy (AFM). In some embodiments, presence of active agent on at least a region of the surface of the coating is determined by X-ray spectroscopy. In some embodiments, presence of active agent on at least a region of the surface of the coating is determined by electronic microscopy. In some embodiments, presence of active agent on at least a region of the surface of the coating is determined by Raman spectroscopy.

In some embodiments, between 25% and 45% of the total amount of active agent in the coating is released after 24 hours in vitro elution in a 1:1 spectroscopic grade ethanol (95%)/phosphate buffer saline at pH 7.4 and 37° C.; wherein the amount of the active agent released is determined by measuring UV absorption at 278 nm by a diode array spectrometer.

In some embodiments, the active agent is at least 50% crystalline. In some embodiments, the active agent is at least 75% crystalline. In some embodiments, the active agent is at least 90% crystalline.

In some embodiments, the polymer comprises a PLGA copolymer. In some embodiments, the coating comprises a first PLGA copolymer with a ratio of about 40:60 to about 60:40 and a second PLGA copolymer with a ratio of about 60:40 to about 90:10. In some embodiments, the coating comprises a first PLGA copolymer having a molecular weight of about 10 kD (weight average molecular weight) and a second polymer is a PLGA copolymer having a molecular weight of about 191(D (weight average molecular weight). In some embodiments, the coating comprises a PLGA copolymer having a number average molecular weight of between about 9.51(D and about 25 kD. In some embodiments, the coating comprises a PLGA copolymer having a number average molecular weight of between about 14.5 kD and about 15 kD. In some embodiments, the coating comprises a PLGA copolymer having a number average molecular weight of between about 25 kD and about 31 kD. In some embodiments, the coating comprises a first PLGA copolymer having a molecular weight of about 25 kD (weight average molecular weight) and a second polymer is a PLGA copolymer having a molecular weight of about 31 kD (weight average molecular weight). In some embodiments, the PLGA weight average molecular weight is a final product specification (that is, not as a raw material, but the weight average molecular weight of the PLGA in its final product form on the stent). In some embodiments, the weight average molecular weight of the PLGA on the device as coated and sintered (which may also include sterilization) is, on average, between about 25,000 and about 31,000 Daltons. In some embodiments, the weight average molecular weight of the PLGA on the device as coated and sintered (which may also include sterilization) is between about 25,000 and about 31,000 Daltons. In some embodiments, the weight average molecular weight of the PLGA on the device as coated and sintered (which may also include sterilization) is between 25,000 and 31,000 Daltons. In some embodiments, the weight average molecular weight of the PLGA on the device as coated and sintered (which may also include sterilization) is about 25,000 Daltons. In some embodiments, the weight average molecular weight of the PLGA on the device as coated and sintered (which may also include sterilization) is about 31,000 Daltons. In some embodiments, the weight average molecular weight of the PLGA on the device as coated and sintered (which may also include sterilization) is at least 25,000 Daltons. In some embodiments, the weight average molecular weight of the PLGA on the device as coated and sintered (which may also include sterilization) is at most 31,000 Daltons. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD (weight average molecular weight) on either side of the target 10 kD (weight average molecular weight).

In some embodiments, the bioabsorbable polymer is selected from the group PLGA, PGA poly(glycolide), LPLA poly(1-lactide), DLPLA poly(d1-lactide), PCL poly(e-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid).

In some embodiments, the stent is formed of stainless steel material. In some embodiments, the stent is formed of a material comprising a cobalt chromium alloy. In some embodiments, the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co. In some embodiments, the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co. In some embodiments, the stent is formed from a material comprising L605 alloy. In some embodiments, the stent is formed from a material comprising MP35N alloy. In some embodiments, the stent is formed from a material comprising the following percentages by weight: about 35Ni, about 35Cr, about 20Co, and about 10Mo. In some embodiments, the stent is formed from a material comprising a cobalt chromium nickel alloy. In some embodiments, the stent is formed from a material comprising Elgiloy®/Phynox®. In some embodiments, the stent is formed from a material comprising the following percentages by weight: about 39 to about 41Co, about 19 to about 21Cr, about 14 to about 16Ni, about 6 to about 8Mo, and Balance (Bal.) Fe. In some embodiments, the stent is formed of a material comprising a platinum chromium alloy. In some embodiments, the stent is formed of an alloy as described in U.S. Pat. No. 7,329,383 incorporated in its entirety herein by reference. In some embodiments, the stent is formed of an alloy as described in U.S. patent application Ser. No. 11/780,060 incorporated in its entirety herein by reference. In some embodiments, the stent may be formed of a material comprising stainless steel, 316L stainless steel, BioDur® 108 (UNS S29108), 304L stainless steel, and an alloy including stainless steel and 5-60% by weight of one or more radiopaque elements such as Pt, IR, Au, W, PERSS® as described in U.S. Publication No. 2003/001830 incorporated in its entirety herein by reference, U.S. Publication No. 2002/0144757 incorporated in its entirety herein by reference, and U.S. Publication No. 2003/0077200 incorporated in its entirety herein by reference, nitinol, a nickel-titanium alloy, cobalt alloys, Elgiloy®, L605 alloys, MP35N alloys, titanium, titanium alloys, Ti-6Al-4V, Ti-50Ta, Ti-10Ir, platinum, platinum alloys, niobium, niobium alloys, Nb-1Zr, Co-28Cr-6Mo, tantalum, and tantalum alloys. Other examples of materials are described in U.S. Publication No. 2005/0070990 incorporated in its entirety herein by reference, and U.S. Publication No. 2006/0153729 incorporated in its entirety herein by reference. Other materials include elastic biocompatible metal such as superelastic or pseudo-elastic metal alloys, as described, for example in Schetsky, L. McDonald, “Shape Memory Alloys”, Encyclopedia of Chemical Technology (3d Ed), John Wiley & Sons 1982, vol. 20 pp. 726-736 incorporated herein by reference, and U.S. Publication No. 2004/0143317 incorporated in its entirety herein by reference. As used herein, the term “about,” when referring to a weight percentage of stent material, means variations of any of 0.5%, 1%, 2%, 5%, 10%, 15%, 20%, 25%, 30%, and 50% of the total weight percent (i.e. 100%) on either side (+/−) of the weight percentage, depending on the embodiment. For example, a weight percentage of stent material of 3.0Fe having a variation of 1% ranges from 2.0 to 4.0, which is a range of 1% of the total (100) on either side of the target 3.0.

In some embodiments, the stent has a thickness of from about 50% to about 90% of a total thickness of the device. In some embodiments, the device has a thickness of from about 20 μm to about 500 μm. In some embodiments, the stent has a thickness of from about 50 μm to about 80 μm. In some embodiments, the coating has a total thickness of from about 5 μm to about 50 μm. The coating can be conformal around the struts, isolated on the abluminal side, patterned, or otherwise optimized for the target tissue. As used herein, the term “about” when referring to a device thickness or coating thickness means variations of any of 0.5%, 1%, 2%, 5%, 10%, 15%, 20%, 25%, 30%, and 50%, depending on the embodiment. For non-limiting example, a device thickness of 20 μm having a variation of 10% ranges from 18 μm to 22 μm, which is a range of 10% on either side of the target 20 μm. For non-limiting example, a coating thickness of 100 μm having a variation of 10% ranges from 90 μm to 110 μm, which is a range of 10% on either side of the target 100 μm.

In some embodiments, the device has an active agent content of from about 5 μg to about 500 μg. In some embodiments, the device has an active agent content of from about 100 μg to about 160 μg. As used herein, the term “about” when referring to a active agent (or pharmaceutical agent) content means variations of any of 0.5%, 1%, 2%, 5%, 10%, 15%, 20%, 25%, 30%, and 50%, depending on the embodiment. For non-limiting example, an active agent content of 120 μg having a variation of 10% ranges from 108 μg to 132 μg, which is a range of 10% on either side of the target 120 μg.

In some embodiments, the active agent is selected from rapamycin, a prodrug, a derivative, an analog, a hydrate, an ester, and a salt thereof. In some embodiments, the active agent is selected from one or more of sirolimus, everolimus, zotarolimus and biolimus. In some embodiments, the active agent comprises a macrolide immunosuppressive (limus) drug. In some embodiments, the macrolide immunosuppressive drug comprises one or more of rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rap amycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O—[2-(2-Hydroxy)ethoxy]ethyl-rapamycin, 40-O—[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, myolimus, and salts, derivatives, isomers, racemates, diastereoisomers, prodrugs, hydrate, ester, or analogs thereof.

In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular pharmaceutical agent herein which is at least in part crystalline, it is understood that any of the possible polymorphs of the pharmaceutical agent are contemplated.

Provided herein is a device comprising a stent; and a coating on the stent; wherein the coating comprises at least one polymer and at least one active agent; wherein the active agent is present in crystalline form on at least one region of an outer surface of the coating opposite the stent and wherein between 25% and 50% of the total amount of active agent in the coating is released after 24 hours in vitro elution.

In some embodiments, the polymer comprises a durable polymer. In some embodiments, the polymer comprises a cross-linked durable polymer. Example biocompatible durable polymers include, but are not limited to: polyester, aliphatic polyester, polyanhydride, polyethylene, polyorthoester, polyphosphazene, polyurethane, polycarbonate urethane, aliphatic polycarbonate, silicone, a silicone containing polymer, polyolefin, polyamide, polycaprolactam, polyamide, polyvinyl alcohol, acrylic polymer, acrylate, polystyrene, epoxy, polyethers, celluiosics, expanded polytetrafluoroethylene, phosphorylcholine, polyethyleneyerphthalate, polymethylmethavrylate, poly(ethylmethacrylate/n-butylmethacrylate), parylene C, polyethylene-co-vinyl acetate, polyalkyl methacrylates, polyalkylene-co-vinyl acetate, polyalkylene, polyalkyl siloxanes, polyhydroxyalkanoate, polyfluoroalkoxyphasphazine, poly(styrene-b-isobutylene-b-styrene), poly-butyl methacrylate, poly-byta-diene, and blends, combinations, homopolymers, condensation polymers, alternating, block, dendritic, crosslinked, and copolymers thereof.

In some embodiments, the polymer comprises is at least one of: a fluoropolymer, PVDF-HFP comprising vinylidene fluoride and hexafluoropropylene monomers, PC (phosphorylcholine), Polysulfone, polystyrene-b-isobutylene-b-styrene, PVP (polyvinylpyrrolidone), alkyl methacrylate, vinyl acetate, hydroxyalkyl methacrylate, and alkyl acrylate. In some embodiments, the alkyl methacrylate comprises at least one of methyl methacrylate, ethyl methacrylate, propyl methacrylate, butyl methacrylate, hexyl methacrylate, octyl methacrylate, dodecyl methacrylate, and lauryl methacrylate. In some embodiments, the alkyl acrylate comprises at least one of methyl acrylate, ethyl acrylate, propyl acrylate, butyl acrylate, hexyl acrylate, octyl acrylate, dodecyl acrylates, and lauryl acrylate.

In some embodiments, the coating comprises a plurality of polymers. In some embodiments, the polymers comprise hydrophilic, hydrophobic, and amphiphilic monomers and combinations thereof. In one embodiment, the polymer comprises at least one of a homopolymer, a copolymer and a terpolymer. The homopolymer may comprise a hydrophilic polymer constructed of a hydrophilic monomer selected from the group consisting of poly(vinylpyrrolidone) and poly(hydroxylalkyl methacrylate). The copolymer may comprise comprises a polymer constructed of hydrophilic monomers selected from the group consisting of vinyl acetate, vinylpyrrolidone and hydroxyalkyl methacrylate and hydrophobic monomers selected from the group consisting of alkyl methacrylates including methyl, ethyl, propyl, butyl, hexyl, octyl, dodecyl, and lauryl methacrylate and alkyl acrylates including methyl, ethyl, propyl, butyl, hexyl, octyl, dodecyl, and lauryl acrylate. The terpolymer may comprise a polymer constructed of hydrophilic monomers selected from the group consisting of vinyl acetate and poly(vinylpyrrolidone), and hydrophobic monomers selected from the group consisting of alkyl methacrylates including methyl, ethyl, propyl, butyl, hexyl, octyl, dodecyl, and lauryl methacrylate and alkyl acrylates including methyl, ethyl, propyl, butyl, hexyl, octyl, dodecyl, and lauryl acrylate.

In one embodiment, the polymer comprises three polymers: a terpolymer, a copolymer and a homopolymer. In one such embodiment the terpolymer has the lowest glass transition temperature (Tg), the copolymer has an intermediate Tg and the homopolymer has the highest Tg. In one embodiment the ratio of terpolymer to copolymer to homopolymer is about 40:40:20 to about 88:10:2. In another embodiment, the ratio is about 50:35:15 to about 75:20:5. In one embodiment the ratio is approximately 63:27:10. In such embodiments, the terpolymer has a Tg in the range of about 5° C. to about 25° C., a copolymer has a Tg in the range of about 25° C. to about 40° C. and a homopolymer has a Tg in the range of about 170° C. to about 180° C. In some embodiments, the polymer system comprises a terpolymer (C19) comprising the monomer subunits n-hexyl methacrylate, N-vinylpyrrolidone and vinyl acetate having a Tg of about 10° C. to about 20° C., a copolymer (C10) comprising the monomer subunits n-butyl methacrylacte and vinyl acetate having a Tg of about 30° C. to about 35° C. and a homopolymer comprising polyvinylpyrrolidone having a Tg of about 174° C. As used herein, the term “about,” when referring to a polymer ratio, means variations of any of 0.5%, 1%, 2%, 5%, 10%, 15%, 20%, 25%, 30%, and 50%, depending on the embodiment. For non-limiting example, a ratio of 40:40:20 having a variation of 10% around each of the polymers (e.g. the terpolymer may be 35-45%; the copolymer may be 35-45%, and the homopolymer may be 15 to 25% of the total). As used herein, the term “about,” when referring to a Tg, means variations of any of 0.5%, 1%, 2%, 5%, 10%, 15%, 20%, 25%, 30%, and 50%, depending on the embodiment. For non-limiting example, a Tg of 30° C. having a variation of 10% means a range of Tg from 27° C. to 33° C.

Some embodiments comprise about 63% of C19, about 27% of C10 and about 10% of polyvinyl pyrrolidone (PVP). The C10 polymer is comprised of hydrophobic n-butyl methacrylate to provide adequate hydrophobicity to accommodate the active agent and a small amount of vinyl acetate. The C19 polymer is soft relative to the C10 polymer and is synthesized from a mixture of hydrophobic n-hexyl methacrylate and hydrophilic N-vinyl pyrrolidone and vinyl acetate monomers to provide enhanced biocompatibility. Polyvinyl pyrrolidone (PVP) is a medical grade hydrophilic polymer.

In some embodiments, the polymer is not a polymer selected from: PBMA (poly n-butyl methacrylate), Parylene C, and polyethylene-co-vinyl acetate.

In some embodiments, the polymer comprises a bioabsorbable polymer. In some embodiments, the bioabsorbable polymer is selected from the group PLGA, PGA poly(glycolide), LPLA poly(1-lactide), DLPLA poly(d1-lactide), PCL poly(e-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid).

In some embodiments, in vitro elution is carried out in a 1:1 spectroscopic grade ethanol

(95%)/phosphate buffer saline at pH 7.4 and 37° C.; wherein the amount of active agent released is determined by measuring UV absorption.

In some embodiments, the active agent is at least 50% crystalline. In some embodiments, the active agent is at least 75% crystalline. In some embodiments, the active agent is at least 90% crystalline.

Provided herein is a device comprising a stent; and a plurality of layers that form a laminate coating on said stent; wherein at least one of said layers comprises a bioabsorbable polymer and at least one of said layers comprises one or more active agents; wherein at least a portion of the active agent is in crystalline form.

Provided herein is a device comprising a stent; and a plurality of layers that form a laminate coating on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein at least a portion of the pharmaceutical agent is in crystalline form.

In some embodiments, the device has at least one pharmaceutical agent layer defined by a three-dimensional physical space occupied by crystal particles of said pharmaceutical agent and said three dimensional physical space is free of polymer. In some embodiments, at least some of the crystal particles in said three dimensional physical space defining said at least one pharmaceutical agent layer are in contact with polymer particles present in a polymer layer adjacent to said at least one pharmaceutical agent layer defined by said three-dimensional space free of polymer.

In some embodiments, the plurality of layers comprises a first polymer layer comprising a first bioabsorbable polymer and a second polymer layer comprising a second bioabsorbable polymer, wherein said at least one layer comprising said pharmaceutical agent is between said first polymer layer and said second polymer layer. In some embodiments, first and second bioabsorbable polymers are the same polymer. In some embodiments, the first and second bioabsorbable polymers are different. In some embodiments, the second polymer layer has at least one contact point with at least one particle of said pharmaceutical agent in said pharmaceutical agent layer and said second polymer layer has at least one contact point with said first polymer layer.

In some embodiments, the stent has a stent longitudinal axis; and said second polymer layer has a second polymer layer portion along said stent longitudinal wherein said second layer portion is free of contact with particles of said pharmaceutical agent. In some embodiments, the device has at least one pharmaceutical agent layer defined by a three-dimensional physical space occupied by crystal particles of said pharmaceutical agent and said three dimensional physical space is free of polymer.

The second polymer layer may have a layer portion defined along a longitudinal axis of the stent, said polymer layer portion having a thickness less than said maximum thickness of said second polymer layer; wherein said portion is free of contact with particles of said pharmaceutical agent.

The polymer layer portion may be a sub layer which, at least in part, extends along the abluminal surface of the stent along the longitudinal axis of the stent (where the longitudinal axis of the stent is the central axis of the stent along its tubular length). For example, when a coating is removed from the abluminal surface of the stent, such as when the stent is cut along its length, flattened, and the coating is removed by scraping the coating off using a scalpel, knife or other sharp tool, the coating that is removed (despite having a pattern consistent with the stent pattern) has a layer that can be shown to have the characteristics described herein. This may be shown by sampling multiple locations of the coating that is representative of the entire coating.

Alternatively, and/or additionally, since stents are generally comprised of a series of struts and voids, the methods provided herein advantageously allow for coatings extending around each strut.

The layers of coating are likewise disposed around each strut. Thus, a polymer layer portion may be a layer which, at least, extends around each strut a distance from said strut (although the distance may vary where the coating thickness on the abluminal surface is different than the coating thickness on the luminal and/or sidewalls).

In some embodiments, the stent comprises at least one strut having a strut length along said stent longitudinal axis, wherein said second layer portion extends substantially along said strut length. In some embodiments, the stent has a stent length along said stent longitudinal axis and said second layer portion extends substantially along said stent length.

In some embodiments, the stent comprises at least five struts, each strut having a strut length along said stent longitudinal axis, wherein said second layer portion extends substantially along substantially the strut length of at least two struts. In some embodiments, the stent comprises at least five struts, each strut having a strut length along said stent longitudinal axis, wherein said second layer portion extends substantially along substantially the strut length of at least three struts. In some embodiments, the stent comprises at least five struts, each strut having a strut length along said stent longitudinal axis, wherein said second layer portion extends substantially along substantially the strut length of least four struts. In some embodiments, the stent comprises at least five struts, each strut having a strut length along said stent longitudinal axis, wherein said second layer portion extends substantially along substantially the strut length of all said at least five struts. In some embodiments, the stent has a stent length along said stent longitudinal axis and said second layer portion extends substantially along said stent length.

In some embodiments, the stent has a stent length along said stent longitudinal axis and said second layer portion extends along at least 50% of said stent length. In some embodiments, the stent has a stent length along said stent longitudinal axis and said second layer portion extends along at least 75% of said stent length. In some embodiments, the stent has a stent length along said stent longitudinal axis and said second layer portion extends along at least 85% of said stent length. In some embodiments, the stent has a stent length along said stent longitudinal axis and said second layer portion extends along at least 90% of said stent length. In some embodiments, the stent has a stent length along said stent longitudinal axis and said second layer portion extends along at least 99% of said stent length.

In some embodiments, the laminate coating has a total thickness and said second polymer layer portion has a thickness of from about 0.01% to about 10% of the total thickness of said laminate coating. In some embodiments, the laminate coating has a total thickness and said horizontal second polymer layer portion has a thickness of from about 1% to about 5% of the total thickness of said laminate coating. In some embodiments, the laminate coating has a total thickness of from about 5 μm to about 50 μm and said horizontal second polymer layer portion has a thickness of from about 0.001 μm to about 5 μm. In some embodiments, the laminate coating has a total thickness of from about 10 μm to about 20 μm and said second polymer layer portion has a thickness of from about 0.01 μm to about 5 μm. As used herein, the term “about” when referring to a laminate coating thickness means variations of any of 0.5%, 1%, 2%, 5%, 10%, 15%, 20%, 25%, 30%, and 50%, depending on the embodiment. For non-limiting example, a laminate coating thickness of 20 μm having a variation of 10% ranges from 18 μm to 22 μm, which is a range of 10% on either side of the target 20 μm. For non-limiting example, a layer portion having a thickness that is 1% of the total thickness of the laminate coating and having a variation of 0.5% means the layer portion may be from 0.5% to 1.5% of the total thickness of the laminate coating thickness. The coating can be conformal around the struts, isolated on the abluminal side, patterned, or otherwise optimized for the target tissue.

In some embodiments, the laminate coating is at least 25% by volume pharmaceutical agent. In some embodiments, the laminate coating is at least 35% by volume pharmaceutical agent. In some embodiments, the laminate coating is about 50% by volume pharmaceutical agent.

In some embodiments, at least a portion of the pharmaceutical agent is present in a phase separate from one or more phases formed by said polymer.

In some embodiments, the pharmaceutical agent is at least 50% crystalline. In some embodiments, the pharmaceutical agent is at least 75% crystalline. In some embodiments, the pharmaceutical agent is at least 90% crystalline. In some embodiments, the pharmaceutical agent is at least 95% crystalline. In some embodiments, the pharmaceutical agent is at least 99% crystalline.

In some embodiments, the stent has a stent longitudinal length and the coating has a coating outer surface along said stent longitudinal length, wherein said coating comprises pharmaceutical agent in crystalline form present in the coating below said coating outer surface. In some embodiments, the stent has a stent longitudinal length and the coating has a coating outer surface along said stent longitudinal length, wherein said coating comprises pharmaceutical agent in crystalline form present in the coating up to at least 1 μm below said coating outer surface. In some embodiments, the stent has a stent longitudinal length and the coating has a coating outer surface along said stent longitudinal length, wherein said coating comprises pharmaceutical agent in crystalline form present in the coating up to at least 5 μm below said coating outer surface.

In some embodiments, the coating exhibits an X-ray spectrum showing the presence of said pharmaceutical agent in crystalline form. In some embodiments, the coating exhibits a Raman spectrum showing the presence of said pharmaceutical agent in crystalline form. In some embodiments, the coating exhibits a Differential Scanning calorimetry (DSC) curve showing the presence of said pharmaceutical agent in crystalline form. In some embodiments, said coating exhibits Wide Angle X-ray Scattering (WAXS) spectrum showing the presence of said pharmaceutical agent in crystalline form. In some embodiments, the coating exhibits a wide angle radiation scattering spectrum showing the presence of said pharmaceutical agent in crystalline form. In some embodiments, the coating exhibits an Infra Red (IR) spectrum showing the presence of said pharmaceutical agent in crystalline form.

In some embodiments, the stent has a stent longitudinal axis and a stent length along said stent longitudinal axis, wherein said coating is conformal to the stent along substantially said stent length.

In some embodiments, the stent has a stent longitudinal axis and a stent length along said stent longitudinal axis, wherein said coating is conformal to the stent along at least 75% of said stent length. In some embodiments, the stent has a stent longitudinal axis and a stent length along said stent longitudinal axis, wherein said coating is conformal to the stent along at least 85% of said stent length. In some embodiments, the stent has a stent longitudinal axis and a stent length along said stent longitudinal axis, wherein said coating is conformal to the stent along at least 90% of said stent length. In some embodiments, the stent has a stent longitudinal axis and a stent length along said stent longitudinal axis, wherein said coating is conformal to the stent along at least 95% of said stent length. In some embodiments, the stent has a stent longitudinal axis and a stent length along said stent longitudinal axis, wherein said coating is conformal to the stent along at least 99% of said stent length.

In some embodiments, the stent has a stent longitudinal axis and a plurality of struts along said stent longitudinal axis, wherein said coating is conformal to at least 50% of said struts. In some embodiments, the stent has a stent longitudinal axis and a plurality of struts along said stent longitudinal axis, wherein said coating is conformal to at least 75% of said struts. In some embodiments, the stent has a stent longitudinal axis and a plurality of struts along said stent longitudinal axis, wherein said coating is conformal to at least 90% of said struts. In some embodiments, the stent has a stent longitudinal axis and a plurality of struts along said stent longitudinal axis, wherein said coating is conformal to at least 99% of said struts. In some embodiments, the stent has a stent longitudinal axis and a stent length along said stent longitudinal axis, wherein an electron microscopy examination of the device shows said coating is conformal to said stent along at least 90% of said stent length.

In some embodiments, the stent has a stent longitudinal axis and a stent length along said stent longitudinal axis, wherein said coating has a substantially uniform thickness along substantially said stent length.

In some embodiments, the stent has a stent longitudinal axis and a stent length along said stent longitudinal axis, wherein said coating has a substantially uniform thickness along at least 75% of said stent length. In some embodiments, the stent has a stent longitudinal axis and a stent length along said stent longitudinal axis, wherein said coating has a substantially uniform thickness along at least 95% of said stent length.

In some embodiments, the stent has a stent longitudinal axis and a stent length along said stent longitudinal axis, wherein said coating has an average thickness determined by an average calculated from coating thickness values measured at a plurality of points along said stent longitudinal axis; wherein a thickness of the coating measured at any point along stent longitudinal axis is from about 75% to about 125% of said average thickness. In some embodiments, the stent has a stent longitudinal axis and a stent length along said stent longitudinal axis, wherein said coating has an average thickness determined by an average calculated from coating thickness values measured at a plurality of points along said stent longitudinal axis; wherein a thickness of the coating measured at any point along stent longitudinal axis is from about 95% to about 105% of said average thickness. As used herein, the term “about” when referring to a coating thickness means variations of any of 0.5%, 1%, 2%, 5%, 10%, 15%, 20%, 25%, 30%, and 50%, depending on the embodiment. For non-limiting example, a coating thickness at a point along the stent longitudinal axis which is 75% of the average thickness and having a variation of 10% may actually be anywhere from 65% to 85% of the average thickness.

Provided herein is a device comprising: a stent; and a plurality of layers that form a laminate coating on said stent, wherein a first layer comprises a first bioabsorbable polymer, a second layer comprises a pharmaceutical agent, a third layer comprises a second bioabsorbable polymer, a fourth layer comprises the pharmaceutical agent, and a fifth layer comprises a third bioabsorbable polymer, wherein the pharmaceutical agent is selected from rapamycin, a prodrug, a derivative, an analog, a hydrate, an ester, and a salt thereof, and wherein at least a portion of the pharmaceutical agent is in crystalline form.

In some embodiments, at least two of said first bioabsorbable polymer, said second bioabsorbable polymer and said third bioabsorbable polymer are the same polymer. In some embodiments, the first bioabsorbable polymer, the second bioabsorbable polymer and the third bioabsorbable polymer are the same polymer. In some embodiments, at least two of said first bioabsorbable polymer, said second bioabsorbable polymer and said third bioabsorbable polymer are different polymers. In some embodiments, the first bioabsorbable polymer, said second bioabsorbable polymer and said third bioabsorbable polymer are different polymers.

In some embodiments, the third layer has at least one contact point with particles of said pharmaceutical agent in said second layer; and said third layer has at least one contact point with said first layer.

In some embodiments, at least two of the first polymer, the second polymer, and the third polymer are the same polymer, and wherein said same polymer comprises a PLGA copolymer. In some embodiments, the third polymer has an in vitro dissolution rate higher than the in vitro dissolution rate of the first polymer. In some embodiments, the third polymer is PLGA copolymer with a ratio of about 40:60 to about 60:40 and the first polymer is a PLGA copolymer with a ratio of about 70:30 to about 90:10. In some embodiments, the third polymer is PLGA copolymer having a molecular weight of about 10 kD (weight average molecular weight) and the second polymer is a PLGA copolymer having a molecular weight of about 19 kD (weight average molecular weight). In some embodiments, the first polymer, the second polymer, and the third polymer each comprise a PLGA copolymer having a number average molecular weight of between about 9.5 kD and about 25 kD. In some embodiments, the first polymer, the second polymer, and the third polymer each comprise a PLGA copolymer having a number average molecular weight of between about 14.5 kD and about 15 kD. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

In some embodiments, measuring the in vitro dissolution rate of said polymers comprises contacting the device with elution media and determining polymer weight loss at one or more selected time points. In some embodiments, measuring the in vitro dissolution rate of said polymers comprises contacting the device with elution media and determining polymer weight loss at one or more selected time points.

Provided herein is a device, comprising: a stent; and a coating on said stent comprising a first bioabsorbable polymer, a second bioabsorbable polymer; and pharmaceutical agent selected from rapamycin, a prodrug, a derivative, an analog, a hydrate, an ester, and a salt thereof wherein at least a portion of the pharmaceutical agent is in crystalline Ruin, and wherein the first polymer has an in vitro dissolution rate higher than the in vitro dissolution rate of the second polymer.

In some embodiments, the first polymer is PLGA copolymer with a ratio of about 40:60 to about 60:40 and the second polymer is a PLGA copolymer with a ratio of about 70:30 to about 90:10. In some embodiments, the first polymer is PLGA copolymer having a molecular weight of about 10 kD (weight average molecular weight) and the second polymer is a PLGA copolymer having a molecular weight of about 19 kD (weight average molecular weight). In some embodiments, the coating comprises a PLGA copolymer having a number average molecular weight of between about 9.5 kD and about 25 kD. In some embodiments, the coating comprises a PLGA copolymer having a number average molecular weight of between about 14.5 kD and about 15 kD. In some embodiments, the coating comprises a PLGA copolymer having a number average molecular weight of between about 25 kD and about 31 kD. In some embodiments, measuring the in vitro dissolution rate of said polymers comprises contacting the device with elution media and determining polymer weight loss at one or more selected time points. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

Provided herein is a device comprising a stent; and a plurality of layers that form a laminate coating on said stent; wherein at least one of said layers comprises a first bioabsorbable polymer, at least one of said layers comprises a second bioabsorbable polymer, and at least one of said layers comprises one or more active agents; wherein at least a portion of the active agent is in crystalline form, and wherein the first polymer has an in vitro dissolution rate higher than the in vitro dissolution rate of the second polymer.

Provided herein is a device comprising a stent; and a plurality of layers that form a laminate coating on said stent; wherein at least one of said layers comprises a first bioabsorbable polymer, at least one of said layers comprises a second bioabsorbable polymer, and at least one of said layers comprises a pharmaceutical agent selected from rapamycin, a prodrug, a derivative, an analog, a hydrate, an ester, and a salt thereof; wherein at least a portion of the pharmaceutical agent is in crystalline form and wherein the first polymer has an in vitro dissolution rate higher than the in vitro dissolution rate of the second polymer.

In some embodiments, the first polymer is PLGA copolymer with a ratio of about 40:60 to about 60:40 and the second polymer is a PLGA copolymer with a ratio of about 70:30 to about 90:10. In some embodiments, the first polymer is PLGA copolymer having a molecular weight of about 10 kD (weight average molecular weight) and the second polymer is a PLGA copolymer having a molecular weight of about 19 kD (weight average molecular weight). In some embodiments, at least one of the first coating and the second coating comprises a PLGA copolymer having a number average molecular weight of between about 9.5 kD and about 25 kD. In some embodiments, at least one of the first coating and the second coating comprises a PLGA copolymer having a number average molecular weight of between about 14.5 kD and about 15 kD. In some embodiments, at least one of the first coating and the second coating comprises a PLGA copolymer having a number average molecular weight of between about 25 kD and about 31 kD. In some embodiments, measuring the in vitro dissolution rate comprises contacting the device with elution media and determining polymer weight loss at one or more selected time points. As used herein, the term “about,” when referring to a copolymer ratio, means variations of any of 0.5%, 1%, 2%, 5%, 10%, 15%, 20%, 25%, 30%, and 50%, depending on the embodiment. For example, a copolymer ratio of 40:60 having a variation of 10% ranges from 35:65 to 45:55, which is a range of 10% of the total (100) about the target. As used herein, the term “about” when referring to a polymer molecular weight means variations of any of 0.5%, 1%, 2%, 5%, 10%, 15%, 20%, 25%, 30%, and 50%, depending on the embodiment. For example, a polymer molecular weight of 10 kD (weight average molecular weight) having a variation of 10% ranges from 9 kD to 11 kD, which is a range of 10% of the target 10 kD on either side of the target 10 kD.

Provided herein is a device comprising a stent; and a plurality of layers that form a laminate coating on said stent; wherein at least one of said layers comprises a bioabsorbable polymer, at least one of said layers comprises a first active agent and at least one of said layers comprises a second active agent; wherein at least a portion of first and/or second active agents is in crystalline form.

In some embodiments, the bioabsorbable polymer is selected from the group PLGA, PGA poly(glycolide), LPLA poly(1-lactide), DLPLA poly(d1-lactide), PCL poly(ε-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid). In some embodiments, the polymer comprises an intimate mixture of two or more polymers.

In some embodiments, the first and second active agents are independently selected from pharmaceutical agents and active biological agents.

In some embodiments, the stent is formed of stainless steel material. In some embodiments, the stent is formed of a material comprising a cobalt chromium alloy. In some embodiments, the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co. In some embodiments, the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co. In some embodiments, the stent is formed from a material comprising L605 alloy. In some embodiments, the stent is formed from a material comprising MP35N alloy. In some embodiments, the stent is formed from a material comprising the following percentages by weight: about 35Ni, about 35Cr, about 20Co, and about 10Mo. In some embodiments, the stent is formed from a material comprising a cobalt chromium nickel alloy. In some embodiments, the stent is formed from a material comprising Elgiloy®/Phynox®. In some embodiments, the stent is formed from a material comprising the following percentages by weight: about 39 to about 41Co, about 19 to about 21Cr, about 14 to about 16Ni, about 6 to about 8Mo, and Balance (Bal.) Fe. In some embodiments, the stent is formed of a material comprising a platinum chromium alloy. In some embodiments, the stent is formed of an alloy as described in U.S. Pat. No. 7,329,383 incorporated in its entirety herein by reference. In some embodiments, the stent is formed of an alloy as described in U.S. patent application Ser. No. 11/780,060 incorporated in its entirety herein by reference. In some embodiments, the stent may be formed of a material comprising stainless steel, 316L stainless steel, BioDur® 108 (UNS S29108), 304L stainless steel, and an alloy including stainless steel and 5-60% by weight of one or more radiopaque elements such as Pt, IR, Au, W, PERSS® as described in U.S. Publication No. 2003/001830 incorporated in its entirety herein by reference, U.S. Publication No. 2002/0144757 incorporated in its entirety herein by reference, and U.S. Publication No. 2003/0077200 incorporated in its entirety herein by reference, nitinol, a nickel-titanium alloy, cobalt alloys, Elgiloy®, L605 alloys, MP35N alloys, titanium, titanium alloys, Ti-6Al-4V, Ti-50Ta, Ti-10Ir, platinum, platinum alloys, niobium, niobium alloys, Nb-1Zr, Co-28Cr-6Mo, tantalum, and tantalum alloys. Other examples of materials are described in U.S. Publication No. 2005/0070990 incorporated in its entirety herein by reference, and U.S. Publication No. 2006/0153729 incorporated in its entirety herein by reference. Other materials include elastic biocompatible metal such as superelastic or pseudo-elastic metal alloys, as described, for example in Schetsky, L. McDonald, “Shape Memory Alloys”, Encyclopedia of Chemical Technology (3d Ed), John Wiley & Sons 1982, vol. 20 pp. 726-736 incorporated herein by reference, and U.S. Publication No. 2004/0143317 incorporated in its entirety herein by reference. As used herein, the term “about,” when referring to a weight percentage of stent material, means variations of any of 0.5%, 1%, 2%, 5%, 10%, 15%, 20%, 25%, 30%, and 50% of the total weight percent (i.e. 100%) on either side (+/−) of the weight percentage, depending on the embodiment. For example, a weight percentage of stent material of 3.0Fe having a variation of 1% ranges from 2.0 to 4.0, which is a range of 1% of the total (100) on either side of the target 3.0.

In some embodiments, the stent has a thickness of from about 50% to about 90% of a total thickness of said device. In some embodiments, the device has a thickness of from about 20 μm to about 500 μm. In some embodiments, the device has a thickness of about 90 μm or less. In some embodiments, the laminate coating has a thickness of from about 5 μm to about 50 μm. In some embodiments, the laminate coating has a thickness of from about 10 μm to about 20 μm. In some embodiments, the stent has a thickness of from about 50 μm to about 80 μm. As used herein, the term “about” when referring to a device thickness or coating thickness or laminate coating thickness means variations of any of 0.5%, 1%, 2%, 5%, 10%, 15%, 20%, 25%, 30%, and 50%, depending on the embodiment. For non-limiting example, a device thickness of 20 μm having a variation of 10% ranges from 18 μm to 22 μm, which is a range of 10% on either side of the target 20 μm. The coating can be conformal around the struts, isolated on the abluminal side, patterned, or otherwise optimized for the particular target tissue.

Provided herein is a device comprising: a stent, wherein the stent is formed from a material comprising the following percentages by weight: 0.05-0.15C, 1.00-2.00Mn, 0.040Si, 0.030P, 0.3S, 19.00-21.00Cr, 9.00-11.00Ni, 14.00-16.00 W, 3.00Fe, and Bal. Co; and a plurality of layers that form a laminate coating on said stent, wherein a first layer comprises a first bioabsorbable polymer, a second layer comprises a pharmaceutical agent, a third layer comprises a second bioabsorbable polymer, a fourth layer comprises the pharmaceutical agent, and a fifth layer comprises a third bioabsorbable polymer, wherein the pharmaceutical agent is selected from rapamycin, a prodrug, a derivative, an analog, a hydrate, an ester, and a salt thereof, wherein at least a portion of the pharmaceutical agent is in crystalline form, and wherein at least one of said first polymer, second polymer and third polymer comprises a PLGA copolymer.

In some embodiments, the device has a pharmaceutical agent content of from about 0.5 μg/mm to about 20 μg/mm In some embodiments, the device has a pharmaceutical agent content of from about 8 μg/mm to about 12 μg/mm. In some embodiments, the device has a pharmaceutical agent content of from about 5 μg to about 500 μg. In some embodiments, the device has a pharmaceutical agent content of from about 100 μg to about 160 μg. As used herein, the term “about” when referring to a active agent content (or pharmaceutical agent content) means variations of any of 0.5%, 1%, 2%, 5%, 10%, 15%, 20%, 25%, 30%, and 50%, depending on the embodiment. For non-limiting example, an active agent content (or pharmaceutical agent content) of 120 μg having a variation of 10% ranges from 108 μg to 132 μg, which is a range of 10% on either side of the target 120 μg. Where content is expressed herein in units of μg/mm, however, this may simply be converted to μg/mm² or another amount per area (e.g., μg/cm2), or vice versa. Similarly, where content is expressed in terms of μg, this may be simply converted to a per-area or per-length term, or vice versa as needed.

Provided herein is a method of preparing a device comprising a stent and a plurality of layers that form a laminate coating on said stent; said method comprising: (a) providing a stent; (b) forming a plurality of layers on said stent to form said laminate coating on said stent; wherein at least one of said layers comprises a bioabsorbable polymer and at least one of said layers comprises one or more active agents; wherein at least a portion of the active agent is in crystalline form.

Provided herein is a method of preparing a device comprising a stent and a plurality of layers that form a laminate coating on said stent; said method comprising: (a) providing a stent; (b) forming a plurality of layers to form said laminate coating on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein at least a portion of the pharmaceutical agent is in crystalline form.

Provided herein is a method of preparing a device comprising a stent and a plurality of layers that form a laminate coating on said stent; said method comprising: (a) providing a stent; (b) forming a plurality of layers to form said laminate coating on said stent; wherein at least one of said layers comprises a bioabsorbable polymer and at least one of said layers comprises a pharmaceutical agent selected from rapamycin, a prodrug, a derivative, an analog, a hydrate, an ester, and a salt thereof; wherein at least a portion of the pharmaceutical agent is in crystalline form, wherein said method comprises forming at least one pharmaceutical agent layer defined by a three-dimensional physical space occupied by crystal particles of said pharmaceutical agent and said three dimensional physical space is free of polymer.

Provided herein is a method of preparing a device comprising a stent and a plurality of layers that form a laminate coating on said stent; said method comprising: (a) providing a stent; (b) discharging at least one pharmaceutical agent and/or at least one active biological agent in dry powder form through a first orifice; (c) forming a supercritical or near supercritical fluid solution comprising at least one supercritical fluid solvent and at least one polymer and discharging said supercritical or near supercritical fluid solution through a second orifice under conditions sufficient to form solid particles of the polymer; (d) depositing the polymer and pharmaceutical agent and/or active biological agent particles onto said substrate, wherein an electrical potential is maintained between the substrate and the polymer and pharmaceutical agent and/or active biological agent particles, thereby forming said coating; and (e) sintering said polymer under conditions that do not substantially modify a morphology of said pharmaceutical agent and/or activity of said biological agent.

In some embodiments, step (b) comprises discharging a pharmaceutical agent selected from rapamycin, a prodrug, a derivative, an analog, a hydrate, an ester, and a salt thereof; wherein at least a portion of the pharmaceutical agent is in crystalline form. In some embodiments, step (c) comprises forming solid particles of a bioabsorbable polymer.

In some embodiments, step (e) comprises forming a polymer layer having a length along a horizontal axis of said device wherein said polymer layer has a layer portion along said length, wherein said layer portion is free of pharmaceutical agent.

In some embodiments, step (e) comprises contacting said polymer with a densified fluid. In some embodiments, step (e) comprises contacting said polymer with a densified fluid for a period of time at a temperature of from about 5° C. and 150° C. and a pressure of from about 10 psi to about 500 psi.

In some embodiments, step (e) comprises contacting said polymer with a densified fluid for a period of time at a temperature of from about 25° C. and 95° C. and a pressure of from about 25 psi to about 100 psi. In some embodiments, step (e) comprises contacting said polymer with a densified fluid for a period of time at a temperature of from about 50° C. and 85° C. and a pressure of from about 35 psi to about 65 psi. The term “about” when used in reference to a temperature in the coating process means variations of any of 0.5%, 1%, 2%, 5%, 10%, 15%, 20%, 25%, 30%, and 50%, on either side of the target or on a single side of the target, depending on the embodiment. For non-limiting example, for a temperature of 150° C. having a variability of 10% on either side of the target (of 150° C.), the temperature would range from 135° C. to 165° C. The term “about” when used in reference to a pressure in the coating process means variations of any of 0.5%, 1%, 2%, 5%, 10%, 15%, 20%, 25%, 30%, and 50%, depending on the embodiment. For non-limiting example, for a pressure of 100 psi having a variability of 10% on either side of the target (of 100 psi), the pressure would range from 90 psi to 110 psi.

Provided herein is a method of preparing a device comprising a stent and a plurality of layers that form a laminate coating on said stent; said method comprising: (a) providing a stent; (b) forming a supercritical or near supercritical fluid solution comprising at least one supercritical fluid solvent and a first polymer, discharging said supercritical or near supercritical fluid solution under conditions sufficient to form solid particles of said first polymer, depositing said first polymer particles onto said stent, wherein an electrical potential is maintained between the stent and the first polymer, and sintering said first polymer; (c) depositing pharmaceutical agent particles in dry powder form onto said stent, wherein an electrical potential is maintained between the stent and said pharmaceutical agent particles; and (d) forming a supercritical or near supercritical fluid solution comprising at least one supercritical fluid solvent and a second polymer and discharging said supercritical or near supercritical fluid solution under conditions sufficient to form solid particles of said second polymer, wherein an electrical potential is maintained between the stent and the second polymer, and sintering said second polymer.

In some embodiments, step (c) and step (d) are repeated at least once. In some embodiments, steps (c) and step (d) are repeated 2 to 20 times.

In some embodiments, the pharmaceutical agent is selected from rapamycin, a prodrug, a derivative, an analog, a hydrate, an ester, and a salt thereof; wherein at least a portion of the pharmaceutical agent is in crystalline form. In some embodiments, the first and second polymers are bioabsorbable.

In some embodiments, step (d) comprises forming a polymer layer having a length along a horizontal axis of said device wherein said polymer layer has a layer portion along said length, wherein said layer portion is free of pharmaceutical agent.

In some embodiments, sintering said first and/or sintering said second polymer comprises contacting said first and/or second polymer with a densified fluid.

In some embodiments, the contacting step is carried out for a period of from about 1 minute to about 60 minutes. In some embodiments, the contacting step is carried out for a period of from about 10 minutes to about 30 minutes.

In some embodiments, maintaining said electrical potential between said polymer particles and or pharmaceutical agent particles and said stent comprises maintaining a voltage of from about 5 kvolts to about 100 kvolts. In some embodiments, maintaining said electrical potential between said polymer particles and or pharmaceutical agent particles and said stent comprises maintaining a voltage of from about 20 kvolts to about 30 kvolts.

Provided herein is a device prepared by a process comprising a method as described herein.

Provided herein is method of treating a subject comprising delivering a device as described herein in a body lumen of the subject.

Provided herein is a method of treating a subject comprising delivering in the body of the subject a device comprising: a stent, wherein the stent is formed from a material comprising the following percentages by weight: 0.05-0.15C, 1.00-2.00Mn, 0.040Si, 0.030P, 0.3S, 19.00-21.00Cr, 9.00-11.00 Ni, 14.00-16.00 W, 3.00Fe, and Bal. Co; and a plurality of layers that form a laminate coating on said stent, wherein a first layer comprises a first bioabsorbable polymer, a second layer comprises a pharmaceutical agent, a third layer comprises a second bioabsorbable polymer, a fourth layer comprises the pharmaceutical agent, and a fifth layer comprises a third bioabsorbable polymer, wherein the pharmaceutical agent is selected from rapamycin, a prodrug, a derivative, an analog, a hydrate, an ester, and a salt thereof, wherein at least a portion of the pharmaceutical agent is in crystalline form, and wherein at least one of said first polymer, second polymer and third polymer comprises a PLGA copolymer.

In some embodiments, the device has a pharmaceutical agent content of from about 0.5 μg/mm to about 20 μg/mm In some embodiments, the device has a pharmaceutical agent content of from about 8 μg/mm to about 12 μg/mm. In some embodiments, the device has a pharmaceutical agent content of from about 100 μg to about 160 μg. In some embodiments, the device has a pharmaceutical agent content of from about 120 μg to about 150 μg. As used herein, the term “about” when referring to a pharmaceutical agent content means variations of any of 0.5%, 1%, 2%, 5%, 10%, 15%, 20%, 25%, 30%, and 50%, depending on the embodiment. For non-limiting example, a pharmaceutical agent content of 120 μg having a variation of 10% ranges from 108 μg to 132 μg, which is a range of 10% on either side of the target 120 μg. Where content is expressed herein in units of μg/mm, however, this may simply be converted to μg/mm² or another amount per area (e.g., μg/cm2), or vice versa, or converted to a total pharmaceutical content by multiplying by the area or length as needed.

In some embodiments, the device has an initial pharmaceutical agent amount and the amount of pharmaceutical agent delivered by said device to vessel wall tissue of said subject is higher than the amount of pharmaceutical agent delivered by a conventional drug eluting stent having the same initial pharmaceutical agent content as the initial pharmaceutical agent content of said device. In some embodiments, the amount of pharmaceutical agent delivered by said device to vessel wall tissue of said subject is at least 25% more that the amount of pharmaceutical agent delivered to vessel wall tissue of said subject by said conventional drug eluting stent. In some embodiments, the method comprises treating restenosis in a blood vessel of said the subject. In some embodiments, the subject is selected from a pig, a rabbit and a human

“Vessel wall tissue” as used herein depicts the tissue surrounding the lumen of a vessel, including the endothelium, neointima, tunica media, IEL (internal elastic lamina), EEL (external elastic lamina), and the tunica adventitia.

In one aspect is a device comprising

a. a stent comprising a cobalt-chromium alloy; and

b. a coating on the stent; wherein the coating comprises at least one polymer and at least one crystalline active agent;
wherein the level of active agent degradation after two weeks incubation in a serum-supplemented cell culture medium at 37° C. is significantly reduced for the device as compared to a device comprising a metal cobalt-chromium stent and a coating comprising at least one polymer and at least one amorphous active agent.

In some embodiments is a device comprising

a. a stent comprising a cobalt-chromium alloy; and

b. a coating on the stent; wherein the coating comprises at least one polymer and at least one crystalline active agent;
wherein the level of active agent degradation after two weeks incubation in a serum-supplemented cell culture medium at 37° C. is reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is 3.4 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 5.0 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 4.5 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 4.0 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 3.4 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 3.0 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 2.5 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 2.0 fold reduced for the device as compared to a device comprising a metal cobalt-chromium stent and a coating comprising at least one polymer and at least one amorphous active agent.

In another aspect is a device comprising

a. a stent comprising a cobalt-chromium alloy; and

b. a coating on the stent; wherein the coating comprises at least one polymer and at least one crystalline active agent;
wherein the coating disassociates from the stent following implantation of the device in a first artery of an animal and spreads within the vessel wall creating coating deposits in the neointima.

In some embodiments, the coating softens following implantation of the device in a first artery of an animal and spreads within the vessel wall creating coating deposits in the neointima. In some embodiments, after fourteen days the coating softens and spreads when observed in vitro.

In another aspect is a device comprising

a. a stent comprising a cobalt-chromium alloy; and

b. a coating on the stent; wherein the coating comprises at least one polymer and at least one crystalline active agent;
wherein there are, on average, fewer than twenty inflammatory cells associated with stent struts of the stent at 3 days following implantation of a single stent in a first artery of an animal.

In another aspect is a device comprising

a. a first stent comprising a cobalt-chromium alloy; and

b. a coating on the first stent; wherein the coating comprises at least one polymer and at least one crystalline active agent;
wherein when said device is implanted in an overlapping manner with a second device in a first artery of an animal wherein the second device comprises

a. a second stent comprising a cobalt-chromium alloy; and

b. a coating on the second stent; wherein the coating comprises at least one polymer and at least one crystalline active agent;
there are, on average, fewer than twenty inflammatory cells associated with stent struts of the first stent at 3 days following implantation in the overlapping region of the overlapping devices.

In some embodiments, the crystalline active agent is at least one of: 50% crystalline, at least 60% crystalline, at least 75% crystalline, at least 80% crystalline, at least 85% crystalline, at least 90% crystalline, at least 95% crystalline, at least 96% crystalline, at least 97% crystalline, at least 98% crystalline, at least 99% crystalline. In certain embodiments, the crystalline active agent comprises pharmaceutical agent comprising at least one polymorph of the possible polymorphs of the crystalline structures of the pharmaceutical agent.

In certain embodiments, the polymer comprises a bioabsorbable polymer. In certain embodiments, the polymer comprises PLGA. In certain embodiments, the polymer comprises PLGA with a ratio of about 40:60 to about 60:40. In certain embodiments, the polymer comprises PLGA with a ratio of about 40:60 to about 60:40 and further comprises PLGA with a ratio of about 60:40 to about 90:10. In certain embodiments, the polymer comprises PLGA having a weight average molecular weight of about 25 kD. In certain embodiments, the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a weight average molecular weight of about 25 kD, a PLGA copolymer having a weight average molecular weight of about 311(D, PGA poly(glycolide), LPLA poly(1-lactide), DLPLA poly(d1-lactide), PCL poly(ε-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof.

In certain embodiments, the stent comprises a cobalt-chromium alloy. In certain embodiments, the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co. In certain embodiments, the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co. In certain embodiments, the stent is formed from a material comprising a platinum chromium alloy or magnesium alloy. In certain embodiments, the stent is formed from a material comprising a platinum chromium alloy. In certain embodiments, the stent is formed from a material comprising a magnesium alloy. In some embodiments, the stent is fully absorbable or resorbable.

In some embodiments, the stent has a thickness of from about 50% to about 90% of a total thickness of the device. In some embodiments, the stent has a thickness of about 50% of a total thickness of the device. In some embodiments, the stent has a thickness of about 60% of a total thickness of the device. In some embodiments, the stent has a thickness of about 70% of a total thickness of the device. In some embodiments, the stent has a thickness of about 80% of a total thickness of the device. In some embodiments, the stent has a thickness of about 90% of a total thickness of the device.

In some embodiments, the coating has a total thickness of from about 5 μm to about 50 μm. In some embodiments, the coating has a total thickness of about 5 μm. In some embodiments, the coating has a total thickness of about 10 μm. In some embodiments, the coating has a total thickness of about 15 μm. In some embodiments, the coating has a total thickness of about 20 μm. In some embodiments, the coating has a total thickness of about 25 μm. In some embodiments, the coating has a total thickness of about 30 μm. In some embodiments, the coating has a total thickness of about 35 μm. In some embodiments, the coating has a total thickness of about 40 μm. In some embodiments, the coating has a total thickness of about 45 μm. In some embodiments, the coating has a total thickness of about 50 μm.

In some embodiments, the device has an active agent content of from about 5 μg to about 500 μg. In certain embodiments, the device has an active agent content of from about 100 μg to about 160 μg. In certain embodiments, the device has an active agent content of about 100 μg. In certain embodiments, the device has an active agent content of about 110 μg. In certain embodiments, the device has an active agent content of about 120 μg. In certain embodiments, the device has an active agent content of about 130 μg. In certain embodiments, the device has an active agent content of about 140 μg. In certain embodiments, the device has an active agent content of about 150 μg. In certain embodiments, the device has an active agent content of about 160 μg.

In some embodiments, the active agent comprises a macrolide immunosuppressive (limus) drug. In some embodiments, the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, myolimus, and salts, derivatives, isomers, racemates, diastereoisomers, prodrugs, hydrate, ester, or analogs thereof.

In some embodiments, the active agent is rapamycin (sirolimus).

In some embodiments, the active agent is a pharmaceutical agent. In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular phamaceutical agent herein which is at least in part crystalline, it is understood that any of the possible polymorphs of the pharmaceutical agent are contemplated.

In some embodiments is a device comprising a stent comprising a cobalt-chromium alloy, and a coating on the stent wherein the coating comprises at least one polymer and crystalline sirolimus; wherein the level of active agent degradation after two weeks incubation in a serum-supplemented cell culture medium at 37° C. is significantly reduced for the device as compared to a device comprising a metal cobalt-chromium stent and a coating comprising at least one polymer and amorphous sirolimus. In some embodiments is a device comprising a stent comprising a cobalt-chromium alloy, and a coating on the stent wherein the coating comprises PLGA and crystalline sirolimus; wherein the level of active agent degradation after two weeks incubation in a serum-supplemented cell culture medium at 37° C. is significantly reduced for the device as compared to a device comprising a metal cobalt-chromium stent and a coating comprising PLGA and amorphous sirolimus.

In some embodiments is a device comprising a stent comprising a cobalt-chromium alloy, and a coating on the stent wherein the coating comprises at least one polymer and crystalline sirolimus; wherein the coating disassociates from the stent following implantation of the device in a first artery of an animal and spreads within the vessel wall creating coating deposits in the neointima. In some embodiments is a device comprising a stent comprising a cobalt-chromium alloy, and a coating on the stent wherein the coating comprises PLGA and crystalline sirolimus; wherein the coating disassociates from the stent following implantation of the device in a first artery of an animal and spreads within the vessel wall creating coating deposits in the neointima.

In some embodiments is a device comprising a stent comprising a cobalt-chromium alloy, and a coating on the stent wherein the coating comprises at least one polymer and crystalline sirolimus; wherein there are, on average, fewer than twenty inflammatory cells associated with stent struts of the stent at 3 days following implantation of a single stent in a first artery of an animal. In some embodiments is a device comprising a stent comprising a cobalt-chromium alloy, and a coating on the stent wherein the coating comprises PLGA and crystalline sirolimus; wherein there are, on average, fewer than twenty inflammatory cells associated with stent struts of the stent at 3 days following implantation of a single stent in a first artery of an animal.

In some embodiments is a device comprising a stent comprising a cobalt-chromium alloy, and a coating on the stent wherein the coating comprises at least one polymer and crystalline sirolimus; wherein when said device is implanted in an overlapping manner with a second device in a first artery of an animal wherein the second device comprises a second stent comprising a cobalt-chromium alloy, and a coating on the second stent wherein the coating comprises at least one polymer and crystalline sirolimus; there are, on average, fewer than twenty inflammatory cells associated with stent struts of the first stent at 3 days following implantation in the overlapping region of the overlapping devices. In some embodiments is a device comprising a stent comprising a cobalt-chromium alloy, and a coating on the stent wherein the coating comprises PLGA and crystalline sirolimus; wherein when said device is implanted in an overlapping manner with a second device in a first artery of an animal wherein the second device comprises a second stent comprising a cobalt-chromium alloy, and a coating on the second stent wherein the coating comprises PLGA and crystalline sirolimus; there are, on average, fewer than twenty inflammatory cells associated with stent struts of the first stent at 3 days following implantation in the overlapping region of the overlapping devices.

In another aspect is a method comprising

providing a coated stent comprising
a stent comprising a cobalt-chromium alloy; and
a coating on the stent; wherein the coating comprises at least one polymer and at least crystalline one active agent; and

wherein the level of active agent degradation after two weeks incubation in a serum-supplemented cell culture medium at 37° C. is significantly reduced for the device as compared to a device comprising a metal cobalt-chromium stent and a coating comprising at least one polymer and at least one amorphous active agent.

In some embodiments is a method comprising
a stent comprising a cobalt-chromium alloy; and
a coating on the stent; wherein the coating comprises at least one polymer and at least crystalline one active agent; and

wherein the level of active agent degradation after two weeks incubation in a serum-supplemented cell culture medium at 37° C. is reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is 3.4 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 5.0 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 4.5 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 4.0 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 3.4 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 3.0 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 2.5 fold reduced for the device as compared to a device comprising a metal cobalt-chromium stent and a coating comprising at least one polymer and at least one amorphous active agent. In some embodiments, the level of active agent degradation after two weeks incubation in a serum-supplemented cell culture medium at 37° C. is at least 2.0 fold reduced for the device as compared to a device comprising a metal cobalt-chromium stent and a coating comprising at least one polymer and at least one amorphous active agent.

In another aspect is a method comprising

providing a coated stent comprising
a stent comprising a cobalt-chromium alloy; and
a coating on the stent; wherein the coating comprises at least one polymer and at least one crystalline active agent; and

implanting the coated stent in an animal,

wherein the coating disassociates from the stent following implantation of the device in a first artery of the animal and spreads within the vessel wall creating coating deposits in the neointima.

In another aspect is a method comprising

providing a coated stent comprising
a stent comprising a cobalt-chromium alloy; and
a coating on the stent; wherein the coating comprises at least one polymer and at
least one crystalline active agent; and

implanting the coated stent in an animal,

wherein there are, on average, fewer than twenty inflammatory cells associated with stent struts of the stent at 3 days following implantation of a single stent in a first artery of the animal

In another aspect is a method comprising

providing a coated stent comprising
a stent comprising a cobalt-chromium alloy; and
a coating on the stent; wherein the coating comprises at least one polymer and at
least one crystalline active agent; and

implanting the coated stent in an animal,

wherein when said device is implanted in an overlapping manner with a second device in a first artery of an animal wherein the second device comprises
a. a second stent comprising a cobalt-chromium alloy; and
b. a coating on the second stent; wherein the coating comprises at least one polymer and at least one crystalline active agent;
   there are, on average, fewer than twenty inflammatory cells associated with stent struts of the first stent at 3 days following implantation in the overlapping region of the overlapping devices.

In some embodiments of the method, the crystalline active agent is at least one of: 50% crystalline, at least 60% crystalline, at least 75% crystalline, at least 80% crystalline, at least 85% crystalline, at least 90% crystalline, at least 95% crystalline, at least 96% crystalline, at least 97% crystalline, at least 98% crystalline, at least 99% crystalline. In certain embodiments, the crystalline active agent comprises pharmaceutical agent comprising at least one polymorph of the possible polymorphs of the crystalline structures of the pharmaceutical agent.

In certain embodiments of the method, the polymer comprises a bioabsorbable polymer. In certain embodiments, the polymer comprises PLGA. In certain embodiments, the polymer comprises PLGA with a ratio of about 40:60 to about 60:40. In certain embodiments, the polymer comprises PLGA with a ratio of about 40:60 to about 60:40 and further comprises PLGA with a ratio of about 60:40 to about 90:10. In certain embodiments, the polymer comprises PLGA having a weight average molecular weight of about 25 kD. In certain embodiments, the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a weight average molecular weight of about 25 kD, a PLGA copolymer having a weight average molecular weight of about 31 kD, PGA poly(glycolide), LPLA poly(1-lactide), DLPLA poly(d1-lactide), PCL poly(e-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof.

In certain embodiments of the method, the stent comprises a cobalt-chromium alloy. In certain embodiments, the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co. In certain embodiments, the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co. In certain embodiments, the stent is formed from a material comprising a platinum chromium alloy or magnesium alloy. In certain embodiments, the stent is formed from a material comprising a platinum chromium alloy. In certain embodiments, the stent is formed from a material comprising a magnesium alloy. In some embodiments, the stent is fully absorbable or resorbable.

In some embodiments of the method, the stent has a thickness of from about 50% to about 90% of a total thickness of the device. In some embodiments, the stent has a thickness of about 50% total of a thickness of the device. In some embodiments, the stent has a thickness of about 60% of a total thickness of the device. In some embodiments, the stent has a thickness of about 70% of a total thickness of the device. In some embodiments, the stent has a thickness of about 80% of a total thickness of the device. In some embodiments, the stent has a thickness of about 90% of a total thickness of the device.

In some embodiments of the method, the coating has a total thickness of from about 5 μm to about 50 μm. In some embodiments, the coating has a total thickness of about 5 μm. In some embodiments, the coating has a total thickness of about 10 μm. In some embodiments, the coating has a total thickness of about 15 μm. In some embodiments, the coating has a total thickness of about 20 μm. In some embodiments, the coating has a total thickness of about 25 μm. In some embodiments, the coating has a total thickness of about 30 μm. In some embodiments, the coating has a total thickness of about 35 μm. In some embodiments, the coating has a total thickness of about 40 μm. In some embodiments, the coating has a total thickness of about 45 μm. In some embodiments, the coating has a total thickness of about 50 μm.

In some embodiments of the method, the device has an active agent content of from about 5 μg to about 500 μg. In certain embodiments, the device has an active agent content of from about 100 μg to about 160 μg. In certain embodiments, the device has an active agent content of about 100 μg. In certain embodiments, the device has an active agent content of about 110 μg. In certain embodiments, the device has an active agent content of about 120 μg. In certain embodiments, the device has an active agent content of about 130 μg. In certain embodiments, the device has an active agent content of about 140 μg. In certain embodiments, the device has an active agent content of about 150 μg. In certain embodiments, the device has an active agent content of about 160 μg.

In some embodiments of the method, the active agent comprises a macrolide immunosuppressive (limus) drug. In some embodiments, the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, myolimus, and salts, derivatives, isomers, racemates, diastereoisomers, prodrugs, hydrate, ester, or analogs thereof.

In some embodiments of the method, the active agent is rapamycin (sirolimus).

In some embodiments of the method, the active agent is a pharmaceutical agent. In some embodiments, the pharmaceutical agent is, at least in part, crystalline. As used herein, the term crystalline may include any number of the possible polymorphs of the crystalline form of the pharmaceutical agent, including for non-limiting example a single polymorph of the pharmaceutical agent, or a plurality of polymorphs of the pharmaceutical agent. The crystalline pharmaceutical agent (which may include a semi-crystalline form of the pharmaceutical agent, depending on the embodiment) may comprise a single polymorph of the possible polymorphs of the pharmaceutical agent. The crystalline pharmaceutical agent (which may include a semi-crystalline form of the pharmaceutical agent, depending on the embodiment) may comprise a plurality of polymorphs of the possible polymorphs of the crystalline pharmaceutical agent. The polymorph, in some embodiments, is a packing polymorph, which exists as a result of difference in crystal packing as compared to another polymorph of the same crystalline pharmaceutical agent. The polymorph, in some embodiments, is a conformational polymorph, which is conformer of another polymorph of the same crystalline pharmaceutical agent. The polymorph, in some embodiments, is a pseudopolymorph. The polymorph, in some embodiments, is any type of polymorph—that is, the type of polymorph is not limited to only a packing polymorph, conformational polymorph, and/or a pseudopolymorph. When referring to a particular phamaceutical agent herein which is at least in part crystalline, it is understood that any of the possible polymorphs of the pharmaceutical agent are contemplated.

In some embodiments is a method comprising providing a stent comprising a cobalt-chromium alloy, and a coating on the stent wherein the coating comprises at least one polymer and crystalline sirolimus; wherein the level of active agent degradation after two weeks incubation in a serum-supplemented cell culture medium at 37° C. is significantly reduced for the device as compared to a device comprising a metal cobalt-chromium stent and a coating comprising at least one polymer and amorphous sirolimus. In some embodiments is a method comprising providing a device comprising a stent comprising a cobalt-chromium alloy, and a coating on the stent wherein the coating comprises PLGA and crystalline sirolimus; wherein the level of active agent degradation after two weeks incubation in a serum-supplemented cell culture medium at 37° C. is significantly reduced for the device as compared to a device comprising a metal cobalt-chromium stent and a coating comprising PLGA and amorphous sirolimus.

In some embodiments is a method comprising providing a stent comprising a cobalt-chromium alloy, and a coating on the stent wherein the coating comprises at least one polymer and crystalline sirolimus; and implanting the coated stent in an animal; wherein the coating disassociates from the stent following implantation of the device in a first artery of an animal and spreads within the vessel wall creating coating deposits in the neointima. In some embodiments is a method comprising providing a stent comprising a cobalt-chromium alloy, and a coating on the stent wherein the coating comprises PLGA and crystalline sirolimus; and implanting the coated stent in an animal; wherein the coating disassociates from the stent following implantation of the device in a first artery of an animal and spreads within the vessel wall creating coating deposits in the neointima.

In some embodiments is a method comprising providing a stent comprising a cobalt-chromium alloy, and a coating on the stent wherein the coating comprises at least one polymer and crystalline sirolimus; and implanting the coated stent in an animal; wherein there are, on average, fewer than twenty inflammatory cells associated with stent struts of the stent at 3 days following implantation of a single stent in a first artery of an animal. In some embodiments is a method comprising a stent comprising a cobalt-chromium alloy, and a coating on the stent wherein the coating comprises PLGA and crystalline sirolimus; and implanting the coated stent in an animal; wherein there are, on average, fewer than twenty inflammatory cells associated with stent struts of the stent at 3 days following implantation of a single stent in a first artery of an animal.

In some embodiments is a method comprising providing a stent comprising a cobalt-chromium alloy, and a coating on the stent wherein the coating comprises at least one polymer and crystalline sirolimus; and implanting the coated stent in an animal; wherein when said device is implanted in an overlapping manner with a second device in a first artery of an animal wherein the second device comprises a second stent comprising a cobalt-chromium alloy, and a coating on the second stent wherein the coating comprises at least one polymer and crystalline sirolimus; there are, on average, fewer than twenty inflammatory cells associated with stent struts of the first stent at 3 days following implantation in the overlapping region of the overlapping devices. In some embodiments is a method comprising providing a stent comprising a cobalt-chromium alloy, and a coating on the stent wherein the coating comprises PLGA and crystalline sirolimus; and implanting the coated stent in an animal; wherein when said device is implanted in an overlapping manner with a second device in a first artery of an animal wherein the second device comprises a second stent comprising a cobalt-chromium alloy, and a coating on the second stent wherein the coating comprises PLGA and crystalline sirolimus; there are, on average, fewer than twenty inflammatory cells associated with stent struts of the first stent at 3 days following implantation in the overlapping region of the overlapping devices.

### EXAMPLES

The following examples are provided to illustrate selected embodiments. They should not be considered as limiting the scope of the invention, but merely as being illustrative and representative thereof. For each example listed below, multiple analytical techniques may be provided. Any single technique of the multiple techniques listed may be sufficient to show the parameter and/or characteristic being tested, or any combination of techniques may be used to show such parameter and/or characteristic. Those skilled in the art will be familiar with a wide range of analytical techniques for the characterization of drug/polymer coatings. Techniques presented here, but not limited to, may be used to additionally and/or alternatively characterize specific properties of the coatings with variations and adjustments employed which would be obvious to those skilled in the art.

### Sample Preparation

Coatings on stents, on coupons, or samples prepared for in-vivo models are prepared as below. Modifications for a given analytical method are presented within the examples shown, and/or would be obvious to one having skill in the art. Thus, numerous variations, changes, and substitutions will now occur to those skilled in the art without departing from the invention. It should be understood that various alternatives to the embodiments of the invention described herein and examples provided may be employed in practicing the invention and showing the parameters and/or characteristics described.

### AC-SES

An absorbable coating-sirolimus-eluting stent (AC-SES) was based on a cobalt-chromium stent platform (GENIUS® Magic, Eurocor GmbH, Bonn Germany), also referred to herein as a Sirolimus DES. The stent has thin 0.0025 inch struts. The coating consists of sirolimus combined with an absorbable polymer, poly(lactide-co-glycolic acid) (PLGA). Unique to this DES is the combination of the absorbable polymer and drug components using a dry powder electrostatic coating process.

### Coatings on Stents

Coated stents were prepared as follows: coated stents comprise a coating deposited on the stent by deposition of rapamycin in dry powder form by RESS methods and equipment described herein and deposition of polymer particles by RESS methods and equipment described herein. A PDPDP (Polymer, sinter, Drug, Polymer, sinter, Drug, Polymer, sinter) coating sequence was used wherein the polymer was 50:50 PLGA, and the drug was rapamycin. The sinter step was performed at 100° C./150 psi/10 min after each “P” (or polymer) layer. There was 135 micrograms +/−15% sirolimus on each coated stent in this study. The coating was about 5-15 micrometers thick on each stent, and comprised a thicker coating on the abluminal surface (coating bias). The coating encapsuled each of the stents.

In some examples, the coated stents have a targeted thickness of ˜15 microns (which includes a mass fraction and/or weight fraction of active agent that is about 25% to about 30% of the total volume of the coating and/or mass of the coating). In some examples, the coating process is PDPDP (Polymer, sinter, Drug, Polymer, sinter, Drug, Polymer, sinter) using deposition of drug in dry powder form and deposition of polymer particles by RESS methods and equipment described herein. In the illustrations below, resulting coated stents may have a 3-layer coating comprising polymer (for example, PLGA) in the first layer, drug (for example, rapamycin) in a second layer and polymer in the third layer, where a portion of the third layer is substantially drug free (e.g. a sub-layer within the third layer having a thickness equal to a fraction of the thickness of the third layer). As described layer, the middle layer (or drug layer) may be overlapping with one or both first (polymer) and third (polymer) layer. The overlap between the drug layer and the polymer layers is defined by extension of polymer material into physical space largely occupied by the drug. The overlap between the drug and polymer layers may relate to partial packing of the drug particles during the formation of the drug layer. When crystal drug particles are deposited on top of the first polymer layer, voids and or gaps may remain between dry crystal particles. The voids and gaps are available to be occupied by particles deposited during the formation of the third (polymer) layer. Some of the particles from the third (polymer) layer may rest in the vicinity of drug particles in the second (drug) layer. When the sintering step is completed for the third (polymer) layer, the third polymer layer particles fuse to form a continuous film that forms the third (polymer) layer.

In some embodiments, the third (polymer) layer however will have a portion along the longitudinal axis of the stent whereby the portion is free of contacts between polymer material and drug particles. The portion of the third layer that is substantially of contact with drug particles can be as thin as 1 nanometer.

In some examples, the stents are made of a cobalt-chromium alloy and are 5 to 50 mm in length, preferably 9 mm to 30 mm in length, with struts of thickness between 20 and 100 microns, preferably 50-70 microns, measuring from an abluminal surface to a luminal surface, or measuring from a side wall to a side wall. In some examples, the stent may be cut lengthwise and opened to lay flat be visualized and/or assayed using the particular analytical technique provided.

The coating may be removed (for example, for analysis of a coating band and/or coating on a strut, and/or coating on the abluminal surface of a flattened stent) by scraping the coating off using a scalpel, knife or other sharp tool. This coating may be sliced into sections which may be turned 90 degrees and visualized using the surface composition techniques presented herein or other techniques known in the art for surface composition analysis (or other characteristics, such as crystallinity, for example). In this way, what was an analysis of coating composition through a depth when the coating was on the stent or as removed from the stent (i.e. a depth from the abluminal surface of the coating to the surface of the removed coating that once contacted the strut or a portion thereof), becomes a surface analysis of the coating which can, for example, show the layers in the slice of coating, at much higher resolution. Coating removed from the stent may be treated the same way, and assayed, visualized, and/or characterized as presented herein using the techniques described and/or other techniques known to a person of skill in the art.

Additional or alternative detail on how the stents (or samples) were prepared for each Example is contained in the respective Example.

### Sample Preparation for In-Vivo Models

Devices comprising stents having coatings disclosed herein are implanted in the porcine coronary arteries of pigs (domestic swine, juvenile farm pigs, or Yucatan miniature swine). Porcine coronary stenting is exploited herein since such model yields results that are comparable to other investigations assaying neointimal hyperplasia in human subjects.

For histopathology and histomorphometry, Yucatan mini-swine were implanted with AC-SES and/or Vision® bare metal stents (BMS, Abbott Vascular, Santa Clara, Calif.) for 3, 30 or 90 days. Stents were deployed in porcine coronary arteries at a balloon:artery ratio of approximately 1.13:1 in either a single stent configuration or in an overlapping stent configuration using same stent pairs (AC-SES or BMS) overlapped by approximately 50%. Eight single stents or stent pairs were used for each stent type and time point.

Additional or alternative detail on how the stents (or samples) were prepared for each Example is contained in the respective Example.

### Histology and Morphometry

At the termination of the in-life portion of the study, porcine hearts were perfusion fixed at 100 mm Hg. Fixed, stented vessels were dissected from the myocardium, sectioned and stained with hematoxylin and eosin as well as Verhoeff's tissue elastin stain. Light microscopy was used to score the tissue for histopathological variables as described in Supplemental Materials. Scoring was performed by a pathologist in a blinded fashion. Inflammation and injury were scored on a per strut basis and the average was calculated per plane and per stent.

Quantitative morphometric analysis may be performed on the histological sections from each stented artery using standard light microscopy and computer-assisted image measurement systems (Olympus MicroSuite Biological Suite). Lumen area, IEL bounded area, stent area and EEL bounded area measured directly. From these measurements, all other morphometric parameters may be calculated.

### Data Acquisition and Analysis

Data was collected as mean+standard error. For selected continuous data, if the assumptions of normality and homogeneity of variance were met, treatment differences were assessed by group t-test. If normality or homogeneity of variance were not met, treatment differences were assessed by Mann-Whitney Rank Sum test. For multiple comparisons, treatment differences were assessed by a Kruskal-Wallis One Way Analysis of Variance on Ranks. For all statistical tests, the null hypothesis of no difference was only rejected if the value of the calculated statistic was less than 0.05 (p<0.05).

### Example 1

### Stent Coating and Stability

A total of six Cypher® DES controls (Cordis, Miami Lakes, Fla.) and twelve AC-SES were used for the stability assessment. The coating was dissolved and extracted from three controls and six AC-SES test articles by addition of 2 ml acetone:methanol (50:50) at baseline for reference to incubated stents. The remaining stents were incubated for 14 days in serum supplemented cell culture media at 37° C. At the end of this period, the stents were removed and their coatings extracted as described above. Extracted samples were stored at −80° C. such that quantification was performed simultaneously on all samples. A fully validated method employing HPLC linked to tandem mass spectrometry (HPLC-MS/MS) was used for analysis of sirolimus and degradants. The analysis was performed using an API 4000 Triple Quad Mass Spectrometer (AB Sciex Instruments) paired to a Waters Acquity HPLC system. Data were captured and analyzed within the Analyst 4.2.1 software package. Sirolimus content was analyzed to quantify relative amount of parent drug and primary degradants.

Maintaining the crystalline structure of sirolimus within the coating conferred enhanced drug stability. When coated stents were incubated in serum-supplemented cell culture medium at 37° C., a reduced percentage of degradants relative to parent drug were found in the stent coatings containing crystalline sirolimus compared to a standard stent coating containing approximately the same amount of sirolimus but in amorphous form. The levels of the major sirolimus degradant two weeks after incubation under simulated use conditions rose from a baseline value of 0.1%±0.06% to 11.0%±1.0% in stents coated with amorphous sirolimus. In stents with coatings incorporating crystalline sirolimus the increase in degradant was much reduced. Here the relative amount of degradant increased from 0.5%±0.5% to 3.2%±1.8%). This 3.4 fold lower value (11%/3.2%) of relative degradant after two weeks incubation represents a statistically significant reduction in sirolimus degradation (p<0.05).

### Example 2

### Coating Dispersion and Drug Delivery

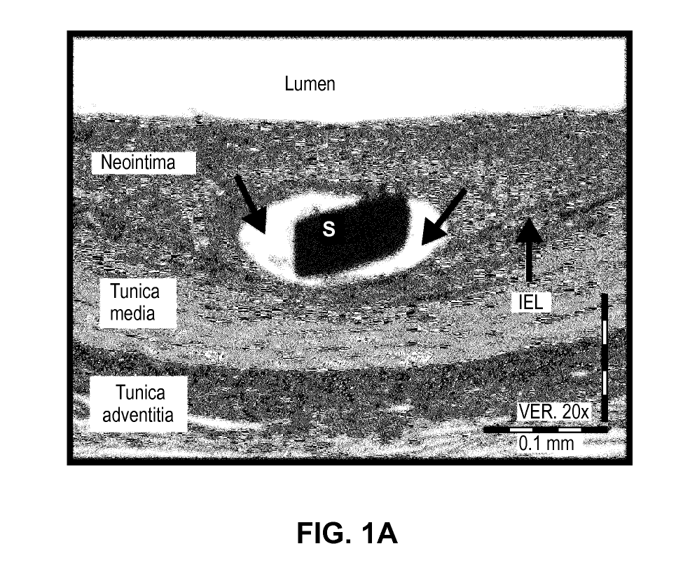

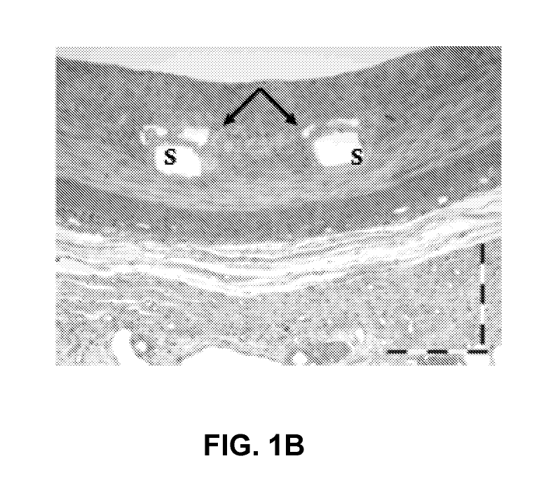

Absorbable coatings behave differently compared to strut-adherent durable polymer coatings. After implantation of the stent, the structure of the absorbable coating enables the polymer material to soften, disassociate from the stent, and spread within the vessel wall creating deposits in the neotima (FIGS. 1A and 1B). Flow loop studies were performed to visualize coating changes in the AC-SES under simulated implant conditions employing pulsatile flow. In these studies, changes in coating morphology were evaluated up to 14 days and the coating was found to become swollen and to gradually spread away from the stent strut in a uniform manner No evidence of delamination was found to occur. The flow loop studies involved a simplified, cell-free preparation where serial visualization of changes in coating morphology is possible. In the flow loop studies, the stent is deployed against the wall of a flexible polymer tube and fluid is pumped through the tubing to hydrate the coating. These studies differ from in vivo implantation into a coronary artery, because in the in vivo situation the stent coating is in direct contact with tissue. As the coating degrades and becomes more porous, cells within the neointima can intercalate into the coating and cause the coating to separate from the stent struts. Most polymer materials, including PLGA, are labile and susceptible to dissolution in the fluids used for histological processing of stented tissue sections. The location, size and shape of the polymer coating in histology sections is witnessed rather as the “negative image” of a space occupying mass. FIGS. 1A and 1B illustrate this phenomenon and provide evidence of coating diassociation from the stent and deposition in the intima after AC-SES implantation into porcine coronary arteries.

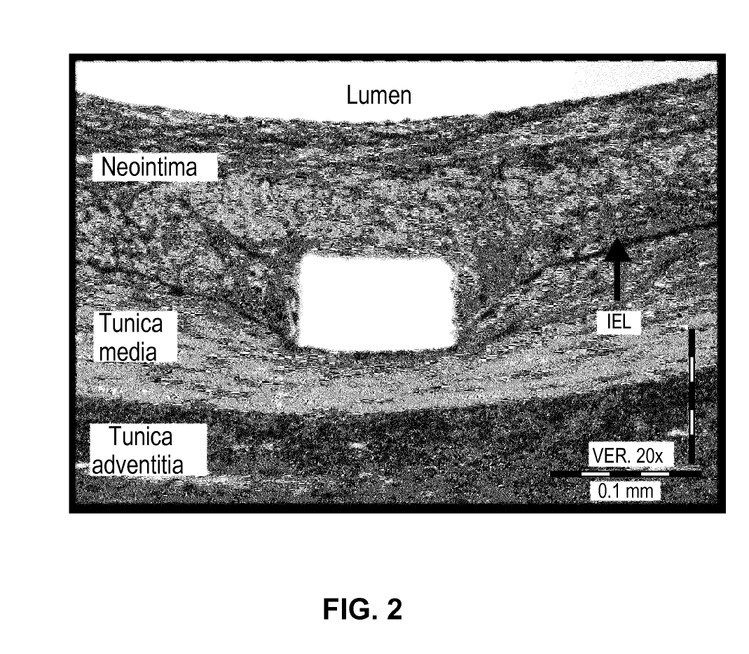

The spreadable coating reduces the load of drug immediately around the stent strut and extends polymer/drug coating from the strut. The coating deposits contain drug still locked in a crystalline lattice combined with the softened PLGA polymer. Thus, the area of drug delivery is increased beyond the immediate vicinity of the stent strut. By 90 days after implantation, there is no further evidence of coating deposits suggesting near complete disassociation of the coating from the stent strut by that point (FIG. 2). Following implantation of any stent coated with an absorbable polymer, the thickness of the strut and its associated coating will vary over time as the polymer absorbs. After 90 days, the absence of stent coating leaves the struts (represented as “S” and seen as the square shaped clear space) at their bare metal dimensions.

The principal advantage of an absorbable stent coating has been viewed as its temporary residence time such that any inflammatory potential brought about by its presence is of limited duration. Shown here is the additional benefit that can be derived from the ability of the absorbable coating to soften, spread and migrate into surrounding tissue. This disassociation of the coating from the stent provides more uniform drug distribution and the ability to saturate binding sites farther from the stent struts. Drug therapy is mediated by this binding to target receptors and it is the amount of drug bound to these receptors at any given time that dictates drug effectiveness, not the total amount of drug present either on the stent or in the artery. As vessel injury from balloon delivery of the stent occurs all along the vessel wall, anti-restenotic therapy is optimized by receptor binding both at and between stent struts. Equally important to the expanded area of drug delivery is the consequent reduction in peak drug concentrations in the immediate vicinity of the stent struts. High concentrations of drug may result in focal regions of tissue toxicity and vessel thrombogenicity (Circulation 112 (2005) 270-278).

### Example 3

### Acute Inflammatory Response

The acute inflammatory response following implantation of sirolimus eluting stent systems processed and created as described herein was evaluated in both single implant and overlapping stent implant configurations and compared to a standard marketed bare metal stent (BMS), the Vision BMS stent (Abbott Vascular). Comparison between two groups of sirolimus eluting stent systems manufactured using two different coating instruments (different coating tool platforms), denoted for this study as “Process 1” or “Automated” and “Process 2” or “Manual”. Process 1 had an automated mechanism to move materials and fixtures through the coating process while Process 2 required manual operation. All other aspects of the coating procedure were the same.

The sirolimus eluting stent systems (Sirolimus DES and systems) were built according to methods described herein, and the coated stents comprised sirolimus and PLGA. The process for making the Sirolimus DES included supercritical fluid deposition which allowed the drug/polymer coating to be applied to a bare metal stent. The absorbable drug/polymer formulation controls drug elution and the duration of polymer exposure. As a result, the coating delivers a therapeutic solution for coronary artery disease with the potential to avoid the long-term safety concerns associated with current drug-eluting coronary stents that use non-absorbing or very slowly absorbing polymers. The sirolimus eluting stents (Sirolimus DES) comprised 3.0×15 mm CoCr stents, having a nominal drug dose per stent of 135 micrograms of sirolimus. Sirolimus DES stents were coated as follows: PDPDP layers (i.e. Polymer Drug Polymer Drug Polymer), having a sinter step (100° C./150 psi/10 min) after each “P” (or polymer) layer, wherein the polymer is 50:50 PLGA. There was 135 micrograms +/−15% sirolimus on each coated stent in this study. The coating was about 5-15 micrometers thick on each stent, and comprised a thicker coating on the abluminal surface (coating bias). The coating encapsuled each of the stents.

The objectives of this study were to evaluate the sirolimus drug eluting stent (Sirolimus DES) produced as described herein, in porcine coronary arteries with respect to acute inflammatory response 3 days after implantation of the stent. A marketed bare metal stent was used as a control. The control stents were 3.0×15 mm CoCr Vision (Abbott Vascular) bare metal stents. Table 1 describes the study design.

TABLE 1

| | | Number of | | |
|---|---|---|---|---|
| | Test/Control | Test/Control | Implantation | Necropsy |
| Group | Articles | Articles | Scheme | Time Point |
| 1 | Sirolimus | n = 7-8 per | Up to 3 | Groups 1V, 3V: |
| | DES | time point | vessels were | Days 3, 30, 90, |
| | (Process 1) | | implanted | and, 180 (±5%) |
| 2 | Sirolimus | n = minimum | per animal | Groups 1, 3: |
| | DES | of 8 per | (RCA, LAD, | Days 3, 30, 90, |
| | (Process 2) | time point | LCX or | 180, and 270 |
| 3 | Vision BMS | n = minimum | branches | (±5%) |
| | (control) | of 8 per | thereof) | Group 2: |
| | | time point | | Day 30 only |
| 1V | Sirolimus | n = minimum | | (±5%) |
| (over- | DES | of 8 pairs per | | |
| lapped) | (Process 1) | time point | | |
| 3V | Vision BMS | n = minimum | | |
| (over- | (control) | of 8 pairs per | | |
| lapped) | | time point | | |

This study enrolled 86 Yucatan pigs (3 and 30 day data from 36 animals are presented herein). Animals underwent a single interventional procedure on Day 0 in which stents were implanted in up to 3 coronary arteries. For Groups 1, 2, and 3: Stents were introduced into the coronary arteries by advancing the stent delivery system (SDS) through the guide catheter and over the guide wire to the deployment site within the coronary artery. The balloon was then inflated at a steady rate to a pressure sufficient to target a visually assessed balloon-artery ratio of 1.05:1-1.15:1. Confirmation of this balloon-artery ratio was made when the angiographic images were quantitatively assessed. After the target balloon-artery ratio was achieved, vacuum was applied to the inflation device in order to deflate the balloon. Complete balloon deflation was verified with fluoroscopy. While maintaining guide wire position, the delivery system was slowly removed. Contrast injections were used to determine device patency and each stent/SDS was evaluated for acute performance characteristics. For Groups 1V, and 3V: Two overlapping stents of the same type were implanted. Each SDS was advanced over the guide wire to the deployment site. The balloon was then inflated at a steady rate to a pressure to target a balloon-artery ratio of 1.05:1-1.15:1. Confirmation of this balloon-artery ratio was made when the angiographic images were quantitatively assessed. After the target balloon-artery ratio was achieved, vacuum was applied to the inflation device in order to deflate the balloon. Complete balloon deflation was verified with fluoroscopy. The delivery system was slowly removed. Any resistance during delivery or removal of the stent delivery system was noted. The second stent of the overlapped pair was advanced to achieve an approximately 50% overlap. Contrast injections were used to determine device patency and each stent/SDS was evaluated for acute performance characteristics. These processes were repeated until stents were deployed in up to 3 vessels.

There were no differences between the Sirolimus DES and Vision BMS controls with respect to device delivery and deployment. Sirolimus DES graded slightly better for trackability. There were few challenges in the swine coronary artery model; however, the proximal Vision BMS in the overlapping stent groups often resisted tracking into the distal stent, which was not observed in the Sirolimus DES groups even with the use of a floppy guidewire. Accuracy of deployment was better with the Sirolimus DES than with the Vision BMS as shown when the Vision BMS would, on occasion, deploy slightly more distal than the target. This was not observed with the Sirolimus DES.

Angiography was performed and recorded on Day 0 (before stent placement, during balloon expansion, and after stent implant) and prior to necropsy. On Day 3 and 30 animals were euthanized and subjected to a comprehensive necropsy and the hearts were collected.

Balloon to artery ratios (ratio of balloon diameter size during peak inflation pressure to the vessel diameter size before stent placement) were calculated from the Quantitative Coronary Angiography (QCA) measurements by dividing the baseline vessel diameter size into the balloon diameter size. Percent stenosis was calculated by subtracting the prenecropsy minimum lumen diameter from the post-implant reference diameter and dividing that value by the post-implant reference diameter. For vessels containing overlapped stents, the proximal and distal stents were averaged to obtain values per vessel. Baseline vessel diameters were similar for all groups of stents at each time point. Average balloon to artery ratios (B:A ratios) for the single Sirolimus DES and single Vision BMS were similar and the overlapping stents were also similar in comparison in both time points. They ranged from approximately 1.09:1 to 1.15:1 which reduces injury to the artery wall and minimizes risk of malapposition. Angiographic evaluation showed there was no difference in mean percent stenosis between any stent groups at either time point. Overall acute performance characteristics and handling of the sirolimus eluting stent & stent systems during implant were comparable to the Vision BMS. Although stent migration did occur, it was infrequent and involved both Sirolimus DES and Vision BMS and always occurred in the proximal LAD where vessel tapering and limited angiographic angles can sometimes affect the accuracy of QCA.

Histopathological scoring via light microscopy was also used to grade the inflammatory response. The inflammation score was determined by the degree and extent of inflammation on a per-strut basis and the average was calculated per plane (i.e., proximal, middle, and distal) and stent. The score was graded as follows: 0 when there were no cells present; 1 for fewer than 20 cells associated with stent strut; 2 when there were greater than 20 cells associated with stent strut, with or without tissue effacement and little to no impact on tissue function; 3 for >20 cells associated with stent strut with effacement of adjacent vascular tissue and adverse impact on tissue function. The acute inflammatory response elicited by the implantation of the Sirolimus DES in both single implant and overlapping stent implant configurations is minimal despite the lack of initial burst of drug release as detailed in Table 2 below.

TABLE 2
three day tissue response
Inflammation Scores following 3 day implantation (range 0-3)

| Stent configuration | Sirolimus DES | Bare Metal Stent |
|---|---|---|
| Single | 0.92 ± 0.13 | 1.00 ± 0.00 |
| Overlapping | 1.00 ± 0.02 | 0.98 ± 0.06 |

The foregoing is illustrative of the present invention, and is not to be construed as limiting thereof. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device comprising
a. a stent comprising a cobalt-chromium alloy; and
b. a coating on the stent; wherein the coating comprises at least one polymer and at least one crystalline active agent;
wherein the level of active agent degradation after two weeks incubation in a serum-supplemented cell culture medium at 37° C. is significantly reduced for the device as compared to a device comprising a metal cobalt-chromium stent and a coating comprising at least one polymer and at least one amorphous active agent.

2. A device comprising
a. a stent comprising a cobalt-chromium alloy; and
b. a coating on the stent; wherein the coating comprises at least one polymer and at least one crystalline active agent;
wherein the coating disassociates from the stent following implantation of the device in a first artery of an animal and spreads within the vessel wall creating coating deposits in the neointima.

3. The device of claim 2,
wherein there are, on average, fewer than twenty inflammatory cells associated with stent struts of the stent at 3 days following implantation of a single stent in a first artery of an animal.

4. The device of claim 2,
wherein when said device is implanted in an overlapping manner with a second device in a first artery of an animal wherein the second device comprises
a. a second stent comprising a cobalt-chromium alloy; and
b. a coating on the second stent; wherein the coating comprises at least one polymer and at least one crystalline active agent;
there are, on average, fewer than twenty inflammatory cells associated with stent struts of the first stent at 3 days following implantation in the overlapping region of the overlapping devices.

5. The device of claim 2, wherein the crystalline active agent is at least one of: 50% crystalline, at least 75% crystalline, at least 90% crystalline.

6. The device of claim 2, wherein the crystalline active agent comprises pharmaceutical agent comprising at least one polymorph of the possible polymorphs of the crystalline structures of the pharmaceutical agent.

7. The device of claim 2, wherein the polymer comprises a bioabsorbable polymer.

8. (canceled)

9. (canceled)

10. (canceled)

11. (canceled)

12. The device of claim 2, wherein the polymer is selected from the group: PLGA, a copolymer comprising PLGA (i.e. a PLGA copolymer), a PLGA copolymer with a ratio of about 40:60 to about 60:40, a PLGA copolymer with a ratio of about 70:30 to about 90:10, a PLGA copolymer having a weight average molecular weight of about 25 kD, a PLGA copolymer having a weight average molecular weight of about 31 kD, PGA poly(glycolide), LPLA poly(1-lactide), DLPLA poly(d1-lactide), PCL poly(e-caprolactone) PDO, poly(dioxolane) PGA-TMC, 85/15 DLPLG p(d1-lactide-co-glycolide), 75/25 DLPLG, 65/35 DLPLG, 50/50 DLPLG, TMC poly(trimethylcarbonate), poly(anhydrides) such as p(CPP:SA) poly(1,3-bis-p-(carboxyphenoxy)propane-co-sebacic acid), and a combination thereof.

13. The device of claim 2, wherein the stent is formed from a material comprising the following percentages by weight: about 0.05 to about 0.15C, about 1.00 to about 2.00Mn, about 0.04Si, about 0.03P, about 0.3S, about 19.0 to about 21.0Cr, about 9.0 to about 11.0Ni, about 14.0 to about 16.00 W, about 3.0Fe, and Bal. Co.

14. The device of claim 2, wherein the stent is formed from a material comprising at most the following percentages by weight: about 0.025C, about 0.15Mn, about 0.15Si, about 0.015P, about 0.01S, about 19.0 to about 21.0Cr, about 33 to about 37Ni, about 9.0 to about 10.5Mo, about 1.0Fe, about 1.0Ti, and Bal. Co.

15. The device of claim 2, wherein the stent is formed from a material comprising a platinum chromium alloy or magnesium alloy.

16. (canceled)

17. The device of claim 2, wherein the stent has a thickness of from about 50% to about 90% of a total thickness of the device.

18. The device of claim 2, wherein the coating has a total thickness of from about 5 μm to about 50 μm.

19. The device of claim 2, wherein the device has an active agent content of from about 5 μg to about 500 μg.

20. (canceled)

21. The device of claim 2, wherein the active agent comprises a macrolide immunosuppressive (limus) drug.

22. The device of claim 21, wherein the macrolide immunosuppressive drug comprises one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycarbonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethylrapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, myolimus, and salts, derivatives, isomers, racemates, diastereoisomers, prodrugs, hydrate, ester, or analogs thereof.

23. The method of claim 24,
wherein the level of active agent degradation after two weeks incubation in a serum-supplemented cell culture medium at 37° C. is significantly reduced for the device as compared to a device comprising a metal cobalt-chromium stent and a coating comprising at least one polymer and at least one amorphous active agent.

24. A method comprising
providing a device comprising
a stent comprising a cobalt-chromium alloy; and
a coating on the stent; wherein the coating comprises at least one polymer and
at least one crystalline active agent; and
implanting the device in an animal,
wherein the coating disassociates from the stent following implantation of the device in a first artery of the animal and spreads within the vessel wall creating coating deposits in the neointima.

25. The method of claim 24,
wherein there are, on average, fewer than twenty inflammatory cells associated with stent struts of the stent at 3 days following implantation of a single stent in a first artery of the animal.

26. The method of claim 24,
wherein when said device is implanted in an overlapping manner with a second device in a first artery of an animal wherein the second device comprises
a. a second stent comprising a cobalt-chromium alloy; and
b. a coating on the second stent; wherein the coating comprises at least one polymer and at least one crystalline active agent;
there are, on average, fewer than twenty inflammatory cells associated with stent struts of the first stent at 3 days following implantation in the overlapping region of the overlapping devices.

27-44. (canceled)
